(19) United States
(12) Patent Application Publication
(10) Pub. No.: US 2004/0081712 A1
(43) Pub. Date: Apr. 29, 2004

(54) Substance for treatment of non-insulin dependent diabetes mellitus, hypertension and/or the metabolic syndrome

(76) Inventors: Kjeld Hermansen, Ega (DK); Soren Gregersen, Ega (DK); Per Bendix Jeppesen, Ega (DK); Lars H. Hoie, Oslo (NO)

Correspondence Address: WINSTON & STRAWN, PATENT DEPARTMENT, 1400 L STREET, N.W., WASHINGTON, DC, 20005-3502, US

(21) Appl. No.: 10/633,396

(22) Filed: Jul. 31, 2003

Related U.S. Application Data
(63) Continuation of application No. PCT/DK01/00523, filed on Jul. 31, 2001.

(30) Foreign Application Priority Data
Feb. 1, 2001 (WO) PCT/DK01/00075

(51) Int. Cl. A61K 35/78

(52) U.S. Cl. 424/757; 424/725

(57) ABSTRACT

A substance including the chemical structures of bicyclo[3.2.1]octan or the chemical structures of kaurene for the use in a dietary supplementation or as a constituent in a medicament for the treatment of non-insulin dependent diabetes mellitus, hypertension and/or the metabolic syndrome. The unique chemical structures of bicyclo[3.2.1]octan alone or in a kaurene structure provides the substances, such as steviol, isosteviol and stevioside, with the capability of enhancing or potentiating the secretion of insulin in a plasma glucose dependent manner. The substances including these unique chemical structures also have the capability of reducing the glucagon concentration in the blood and/or lowering the blood pressure thereby providing a self-regulatory treatment system for non-insulin dependent diabetes mellitus and/or hypertension. In a combination drug which also comprise a soy protein, and/or fibre and/or at least one phytoestrogen these substances act synergistically and such combination drugs are highly useful both prophylacticly or directly in the treatment of the metabolic syndrome and obesity and has due to the self-regulatory effect a widespread applicability as a dietary supplementation.

### CROSS-REFERENCE TO RELATED APPLICATIONS

This application is a continuation of International application PCT/DK01/00523 filed Jul. 31, 2001, the entire content of which is expressly incorporated herein by reference thereto.

### BACKGROUND

The present invention relates to a new medicament for the treatment of non-insulin dependent diabetes mellitus, hypertension and/or the metabolic syndrome.

The present invention further relates to soy protein, phytoestrogens and dietary fibres and compositions thereof in the combination with a substance including a bicyclo[3.2.1]octan of the structural formula I shown in claim 1 and/or a kaurene structure of the structural formula II shown in claim 2 and suitable for preventing and/or treating type 2 diabetes and/or the metabolic syndrome.

The present invention also pertains to the use of such compositions in the prevention and/or treatment of a cardiovascular disease in a subject suffering from type 2 diabetes. The compositions are particularly useful in preventing and/or reducing the influx of triglycerides and/or cholesterol into the arterial wall of diabetic subjects. The compositions are also useful in lowering serum glucose levels and/or serum levels of cholesterol and/or triglycerides and/or blood pressure in diabetic subjects. The present invention also relates to the use of these compositions as a medicament and/or in the manufacture of a medicament for treating type 2 diabetes and/or the metabolic syndrome and/or a cardiovascular disease in a subject suffering from type 2 diabetes. In addition, the present invention also provides methods for preventing and/or treating and/or prophylactically treating and/or alleviating by therapy said diseases and disorders.

Diabetes is a common disease that has a prevalence of 2 to 4% in the population. Non-insulin dependent diabetes mellitus comprises about 85% of diabetes most commonly occurring at the age above 40 years. The incidence of non-insulin dependent diabetes mellitus is increasing and is at a global level expected to surpass 200 million subjects at year 2010.

Diabetes is associated with increased morbidity and a 24-fold increase in mortality primarily due to cardiovascular diseases and strokes.

Non-insulin dependent diabetes mellitus develops especially in subjects with insulin resistance and a cluster of cardiovascular risk factors such as obesity, hypertension and dyslipidemia, a syndrome which first recently has been recognised and is named “The metabolic syndrome” (Alberti K. G., Zimmet P. Z.; Definition, diagnosis and classification of diabetes mellitus and its complications”. Part 1: Diagnosis and classification of diabetes mellitus provisional report of a WHO consultation. Diabet. Med. 1998 July; 15(7), p. 539-53).

A patient has in accordance with the WHO-definition (www.idi.org.au/whoreport.htm) the metabolic syndrome if insulin resistance and/or glucose intolerance is present together with two or more of the following components

reduced glucose tolerance or diabetes

insulin sensivity (under hyperinsulinaemic, euglycaemic conditions corresponding to a glucose uptake below the lower quartile for the background population)

increased blood pressure (>140/90 mmHg)

increased plasma triglyceride (>1.7 mmol/l) and/or low HDL cholesterol (<0.9 mmol/l for men; <1.0 mmol/l for women)

central adipositas (waist/hip ratio for men: >0.90 and for women>0.85) and/or Body Mass Index>30 kg/m²)

micro albuminuria (urine albumin excretion: >20% g min⁻¹ or albumin/creatinine ratio>2.0 mg/mmol.

It has become increasingly evident that the treatment should aim at simultaneously normalising blood glucose, blood pressure, lipids and body weight to reduce the morbidity and mortality. Diet treatment, exercise and avoiding smoking is the first treatment modalities that should be started. However, it will often be necessary to add pharmacological therapy but until today no single drug that simultaneously attack hyperglycaemia, hypertension and dyslipidemia are available for patients with the metabolic syndrome. Instead, these patients may be treated with a combination of several different drugs in addition to e.g. diet. This type of treatment is difficult to adjust and administer to the patient and such treatment may result in many unwanted adverse effects which in themselves may need medical treatment.

Consequently there is a long felt need for a new and combined medicament for the treatment of the metabolic syndrome thereby also preventing an increase in the number of persons developing the non-insulin dependent diabetes mellitus.

Existing oral antidiabetic medicaments to be used in such treatment include the classic insulinotropic agents sulphonylureas (Lebovitz H. E. 1997. “The oral hypoglycemic agents”. In: Ellenberg and Rifkin's Diabetes Mellitus. D. J. Porte and R. S. Sherwin, Editors: Appleton and Lange, p. 761788).

They act primarily by stimulating the sulphonylurea-receptor on the insulin producing beta-cells via closure of the K⁺ ATP-sensitive channels.

However if such an action also affects the myocytes in the heart, an increased risk of cardiac arrhytmias might be present.

Also, it is well known in the art that sulphonylureas can cause severe and lifethreatening hypoglycemia, due to their continuous action as long as they are present in the blood.

Several attempts to develop new antidiabetic agents and drugs for the treatment or prophylactic treatment of the syndrome not having the adverse effects mentioned above, e.g. hypoglycemia and potential harmful actions on the heart functions have been made over the years.

Consumption of soy protein rather than animal protein has been found to lower blood cholesterol (Anderson J. W., Johnstone B. M., Cook-Newell M. E.: Meta-analysis of the effects of soy protein intake on serum lipids. N. Engl. J. Med. 1995; 333; p. 276-282). In addition to this knowledge, recent research also provides evidence that soy protein and/or isoflavones may improve endothelial function and attenuate events leading to both lesion and thrombus formation (Anderson J. W., Johnstone B. M., Cook-Newell M. E.: “Meta-analysis of the effects of soy protein intake on serum lipids”; N. Engl. J. Med. 1995; 333; p. 276-282; Potter S. M., Soy protein and cardiovascular disease: “The impact of bioactive components in soy”. Nutrition Reviews 1998;56, p. 231-235).

Adlercreutz (Finnish Medical Society, Ann. Med. 29, 95-120 (1997)) stressed that no definite recommendations can be made as to the dietary amounts of soy isoflavones needed for prevention of disease. It is emphasized that phytoestrogens, particularly in association with soy intake, seem to have a great potential for preventing cardiovascular diseases, but as the area is really in the early stages of development, an established beneficial effect of soy and isoflavonoids in this respect will have to await further studies. It is further stated that despite an abundant literature at this early stage of dietary phytoestrogen research, much work is needed before any recommendation as to phytoestrogen consumption can be made. However, experimental and epidemiological evidence does support the view that these compounds do not have any negative effects and that they may form a group of substances with a great potential in preventive medicine. It is emphasized that at present, no definite recommendations can be made as to the dietary amounts needed for disease prevention. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Anderson (N. Eng. J. Med. 333, 276-282(1995)) analysed a total of 38 clinical trials and concluded that the consumption of soy protein significantly decreases serum levels of total cholesterol, LDL-cholesterol and triglycerides. It was found that ingestion of diets containing soy protein, as compared with control diets, was accompanied by a significant reduction in serum concentrations of total cholesterol, LDL-cholesterol and triglycerides. However, soy protein intake did not significantly affect serum HDL-cholesterol concentrations. Various types of soy proteins were studied, such as isolated soy protein, textured soy protein, or a combination thereof, and it was found that the type of soy protein did not have any significant effect on the net change in serum cholesterol levels. The study did not consider a simultaneous intake of the various types of soy proteins along with dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Hendrich (J. Nutr. 124(9 Suppl.), 1789S-1792S (1994)) has reported that isoflavones may be of great potential benefit to human health maintenance and that isoflavones may be health-protective in amounts potentially available from a human diet containing daily soy foods. The food content of isoflavones is in the range of from 0.1 to 1 mg/g in soy foods. Several factors such as variety of soybean, processing and the addition of other ingredients to the food, influence isoflavone contents of foods. It is stated that human intestinal bacteria can destroy ingested isoflavones to a great extent and that this may be why only 15 to 20 percent of isoflavones are reported to be recoverable in intact form from the urine and faeces. It is emphasized that much work remains to determine the relation between concentration of isoflavones in human urine and plasma and the biological effects of the isoflavones. It is noted that although more health-related animal data need to be obtained, the time is approaching when long-term human feeding trials of purified isoflavones and foods containing isoflavones to examine health-related outcomes may be warranted. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Knight (Maturitas 22, 167-175 (1995)) provides a synopsis of the literature relating principally to the clinical effects of phytoestrogens on the diseases associated with ageing. A review of literature pertaining to cardiovascular diseases states that the protective effects of phytoestrogens are manifested through lipid changes, a decrease in LDL-cholesterol and an increase in HDL-cholesterol, and vascular effects, concerning both vasomotor tone and vessel wall compliance. The consumption of soy protein is reported to alter lipid levels and dietary soy protein appears to be anti-atherogenic when compared with various animal proteins. It is concluded that isoflavones represent a large and exciting group of compounds with potential benefits to many diseases including diabetes. It is emphasised that current evidence justifies the conclusion that phytoestrogens may be among the dietary factors affording protective effects against heart disease. However, further clinical studies are required to more clearly elucidate their effects. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Knight (Obstet. Gynecol. 87, 897-904 (1996)) has reviewed the sources, metabolism, potencies, and clinical effects of phytoestrogens on humans. The review suggests that phytoestrogens are among the dietary factors affording protection against heart disease in vegetarians. Based on epidemiological and cell line studies, it is emphasized that intervention studies are now an appropriate consideration to assess the clinical effects of phytoestrogens because of the potentially important health benefits associated with the consumption of foods containing these compounds. It is concluded that clinical applications for phytoestrogens are still in their infancy. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Packard (Arterioscler. Thromb. Vasc. Biol. 17, 3542-3556 (1997)) has reviewed the heterogeneity in the apoB containing lipoprotein classes and provides an interpretation of kinetic studies of apoB metabolism in the light of underlying structural and functional variations. The review is based on the fact that lipoprotein classes are composed of a limited number of components with distinct properties. However, the basis for this heterogeneity and the consequences for disease are not thoroughly understood. The LDL-fraction is made up of a small number of subtypes of particles with relatively discrete size and density. Subjects with a preponderance of small-sized LDL have a three-fold increased risk of having a myocardial infarction independent of the total concentration of LDL present. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Potter (Am. J. Clin. Nutr. 58, 501-506 (1993)) studied the effects of soy protein consumption with and without soy fibre on plasma lipids in mildly hypercholesterolemic men. It was reported that total and LDL-cholesterol concentrations can be lowered significantly in mildly hypercholesterolemic men, as indicated by a replacement of 50 percent of dietary protein with soy protein. Similar reductions in blood lipids were noted for isolated soy protein, whether it was consumed in conjunction with soy cotyledon fibre or cellulose fibre. Plasma triglyceride concentrations were unaffected by the various dietary treatments described in the article. The study did not reveal any additive cholesterol-lowering effect of concurrent intake of cotyledon soy fibre with isolated soy protein, and it was stated that whether or not there is an added benefit in lowering blood cholesterol concentrations from increased concurrent intake of soy protein and fibre in humans is not known. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Reinli (Nutr. Cancer 26, 123-148 (1996)) has reviewed the literature for quantitative data on the levels of known phytoestrogens (daidzein, genistein, coumestrol, formononetin and biochanin A) in food plants. It is reported that the isoflavones daidzein and genistein may exist in four related chemical structures, i.e. an aglycone structure (daidzein and genistein), an 7-O-glucoside structure (daidzin and genistin), an 6′-O-acetylglucoside structure (6′-O-acetyldaidzin and 6′-O-acetylgenistin), and a 6′-O-malonylglucoside structure (6′-O-malonyldaidzin and 6′-O-malonylgenistin). The conjugates (7-O-glucosides, 6′-O-acetylglucosides, and 6′-O-malonylglucosides) are transformed to aglycones, which are sometimes called free isoflavones, through hydrolysis in the intestinal tract by β-glucosidase enzymes of gut bacteria. Acid hydrolysis in the stomach may also contribute to the formation of free isoflavones. It is unclear how readily conjugates undergo intestinal hydrolysis and subsequent absorption. It is stressed that isoflavones are metabolised differently by different animals and humans. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

Sniderman (Am J. Cardiol. 79, 64-67 (1997)) presents a risk factor hypothesis with an emphasis on the integral role of LDL in atherogenesis. It is stressed that a measurement of LDL-cholesterol is an incomplete estimate of the risk attributable to LDL and that other classic risk factors such as e.g. hypertension, diabetes, and smoking exert their proatherogenic potential largely or exclusively by multiplying the malign influences of LDL on the arterial wall. It is acknowledged that small, dense LDL-particles are one of the most common dyslipoproteinaemias associated with coronary artery disease. It is reported that elevated levels of lipoprotein (a) are associated with increased coronary risk, but the basis for this is still not clear. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

WO 95/10512 relates to a process for producing an aglucone isoflavone enriched vegetable protein whey and discloses in one embodiment a whey having a dry basis genistein content of about 2.6 to about 8.7 mg/gram and a dry basis daidzein content of about 2.5 to about 6.0 mg/gram. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

WO 95/10529 relates to a process for producing an aglucone isoflavone enriched protein concentrate and discloses in one embodiment a concentrate having on a dry basis a genistein content of about 1.0 to about 2.0 mg/gram and a daidzein content of about 0.7 to about 1.5 mg/gram. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

WO 95/10530 relates to a process for producing an aqueous extract comprising protein and glucone isoflavones and discloses in one embodiment a vegetable protein isolate having a dry basis genistein content of about 1.5 to about 3.5 mg/gram and a dry basis daidzein content of about 1.0 to about 3.0 mg/gram. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

WO 97/31546 discloses data from total replacement programmes (for 6 weeks) in weight reduction studies conducted at Karolinska Hospital in Sweden. It is shown that products comprising isolated soy protein and soybean cotyledon fibres reduce serum cholesterol levels by a maximum of 27 percent and triglyceride levels by a maximum of 44 percent for a patient population with a mean initial cholesterol content of 5.6 mmol/l. A mean value of 6.25 mmol/l was determined for all patients having serum cholesterol levels above 6 mmol/l, and for this group of patients a reduction in serum cholesterol levels of 33 percent was observed. Since the reported data were part of a weight reduction programme, a dietary effect and/or an effect related to a weight loss would have contributed to the observed reductions in cholesterol and/or triglycerides. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

WO 97/37547 discloses an isoflavone-enriched soy protein product having a protein content greater than 60 percent of total dry matter, a total dietary fibre content of less than 4 percent of total dry matter, a sucrose content greater than 10 percent of total dry matter, a total content of sulphur-containing amino acids greater than 2.2 percent of the total amino acid content, a stachyose content of less than 1.5 percent of total dry matter, and a total isoflavone content greater than 2.5 mg/gram, equivalent to 0.25 percent. The use of soy cotyledon fibres is not anticipated and the claimed invention is for use as an ingredient in the production of an edible product and not in a treatment of diabetes. Also, the product differs from the present invention by comprising total dietary fibre in an amount of less than 4 percent of total dry matter, and by containing an unusually low amount of stachyose and a high amount of sulphur-containing amino acids. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

U.S. Pat. No. 5,320,949 discloses a process for producing an aglucone isoflavone enriched fibre product from a vegetable protein material in the form of a slurry and discloses in one embodiment an aglucone enriched fibre product directly obtainable from said process and having a genistein content of about 1.0 and 2.0 mg/gram and a daidzein content of about 0.7 to 1.7 mg/gram. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

U.S. Pat. No. 5,352,384 discloses an aglucone enriched fibre product having a genistein content of about 1.0 to 2.0 mg/gram and a daidzein content of about 0.7 to 1.7 mg/gram. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

EP 827 698 A2 and EP 827 698 A3 disclose a process for producing an aglucone isoflavone enriched extract from a vegetable material containing isoflavone conjugates and protein. No reference is made to a treatment of diabetes by using a composition comprising a combination of soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

An abstract presented at the American Heart Association's 38^{th} Annual Conference on Cardiovascular Disease Epidemiology and Prevention held in March 1998 disclosed a reduction in the levels of total and LDL-cholesterol in a subject following intake of a diet supplemented with 25 grams of soy protein containing 4 mg, 25 mg, 42 mg, and 58 mg of isoflavones, respectively. A “dose-response” effect was reported so that increasing amounts of isoflavones were associated with an increasing reduction of cholesterol. A maximum reduction of serum levels of total and LDL-cholesterol of 4 percent and 7 percent, respectively, was reported for the product containing 58 mg of isoflavone. No reference is made to a treatment of diabetes by using a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres. No reference is made to a composition comprising a combination of a substance including a bicyclo[3.2.1]octan of the structural formula I shown in claim 1 and/or a kaurene structure of the structural formula II shown in claim 2, soy protein, a high content of a phytoestrogen compound, and dietary fibres.

In the attempt to develop new antidiabetic agents and drugs for the treatment or prophylactic treatment of the metabolic syndrome has plants provide a vast resource of compounds with the potential to become new antidiabetic agents.

For instance extracts of the leaves of Stevia rebaudiana Bertoni, a herbaceous member of the Compositae family, have been used for many years in the treatment of diabetes among Indians in Paraguay and Brazil (Sakaguschi M., Kan P Aspesquisas japonesas com Stevia rebaudiana (Bert) Bertoni e o estevioside. Cienc. Cultur. 34; p. 235-248,1982; Oviedo C. A., Franciani G., Moreno R., et al. “Action hipoglucemiante de la Stevia Rebaudiana Bertoni (Kaa-he-e)”. Excerpt. Med. 209, p. 92,1979; Curi R., Alvarez M., Bazotte R. B., et al. Effect of Stevia rebaudiana on glucose tolerance in normal adult humans. Braz. J. Med. Biol. Res., 19, p. 771-774,1986; Hansson J. R., Oliveira B. H., “Stevioside and related sweet diterpenoid glycoside”. Nat. Prod. Rep. 21, p.301-309,1993).

Also, an antihyperglycemic effect has been found in rats when supplementing the diet with dried S. rebaudiana leaves (Oviedo C. A., Franciani G., Moreno R., et al. “Action hipoglucemiante de la Stevia Rebaudiana Bertoni (Kaa-he-e)”. Excerpt. Med. 209:92,1979).

Curi et al. found a slight suppression of plasma glucose when extracts of Stevia rebaudiana leaves were taken orally during a 3-day period. Furthermore, Oviedo et al. reported that tea prepared from the leaves caused a 35% reduction in blood glucose in man.

A number of Stevia species have been examined and shown to contain labdanes, clerodanes, kaurenes and beyerenes (Hansson J. R., Oliveira B. H., “Stevioside and related sweet diterpenoid glycoside”. Nat. Prod. Rep. 21, p. 301-309,1993). Any of these substances as well as many others unidentified substances in the leaves could be responsible for the reduction in blood glucose in man.

In the work of Malaisse W. J. et al (Malaisse W. J., Vanonderbergen A., Louchami K, Jijakli H. and Malaisse-Lagae F., “Effects of Artificial Sweeteners on Insulin Release and Cationic Fluxes in Rat Pancreatic Islets”, Cell. Signal. Vol 10, No. 10, p. 727-733, 1998) the effect of several artificial sweeteners, including stevioside, on insulin release from isolated normal pancreatic rat islets were studied. In this study it was reported that in the presence of 7 mmol/l D-glucose, stevioside in a concentration of 1,0 mmol/l caused a significant increase in insulin output. Also the control group demonstrated a significant increase in insulin output of about 16 times above the basal release value in the presence of 20 mmol/l D-glucose increase. It is therefore uncertain whether the insulin releasing effect is due to the increased glucose level or the presence of stevioside. No diabetic islet cells were studied and the skilled person within the art will know that the mechanism for stimulating normal pancreatic islet cells either not functions at its optimum or not functions at all in the diabetic pancreatic cells, and that the study provided no certain indication of the possible use of stevioside in the treatment of non-insulin dependent diabetes mellitus, hypertension and/or the metabolic syndrome.

In a Chinese study (Lin Qi-Xian, Cao Hai-Xing, Xie Dong, Li Xing-Ming, Shang Ting-Lan, Chen Ya-Sen, Ju Rui-Fen, Dong Li-Li, Wang Ye-Wen, Quian Bao-Gong, “Experiment of Extraction of Stevioside”, Chinese Journal og Pharmaceuticals 1991, No. 22, p 389-390) is indicated a method for extracting stevioside from stevioside leafs from the origin of Bingzzhou in the Hunan Province. The content of stevioside in the extract was determined using HPLC although the article is silent of the purity of the extract. The produced stevioside tablets were for no apparent reason and medical indication applied to patients in the Wuhan Second Hospital. No data on the influence of stevioside on blood glucose, insulin and/or blood pressure is revealed. It is stated that the tablets were effective to diabetes and hypertension during preliminary clinical observations. However, total lack of data on blood glucose, insulin and/or blood pressure i.e. lack of support by test results and the missing information of which types of diabetes that were treated makes this an unsupported and unconfirmed assertion.

Any detailed information of which substance or substances in the leaves that might cause a possible anti-hyperglycemic effect has not yet been disclosed for certainty, and the mechanism of how and to which extent the plasma glucose is reduced is unknown.

The above mentioned articles and studies are concerned with the initial discovery of the effects and provide no evidence of which specific component(s) in the leaves that might be the active one(s).

The effect of intravenous stevioside on the blood pressure was studied in spontaneously hypertensive rats (“The Effect of Stevioside on Blood Pressure and Plasma Catecholamines in Spontaneously Hypertensive Rats”, Paul Chan, De-Yi Xu, Ju-Chi Liu, Yi-Jen Chen, Brian Tomlinson, Wen-Pin Huang, Juei-Tang Cheng, Life Science, Vol. 63, No. 19, 1998, p. 1679-1684). The study showed that during an intravenously administration of stevioside of 200 mg/kg the hypotensive effect was at a maximum, but although reported as being significantly, the fall in the systolic blood pressure was only small. Neither the heart rate nor the plasma catecholamines were significantly changed during the observation period. This study indicated that stevioside advantageously could be used for treating hypertension.

None of the above mentioned references which concerns leaves from Stevia species or stevioside, mentions lipid lowering effects. Furthermore, no reports of an effect of stevioside on plasma glucagon level have previously been reported. Glucagon, a pancreatic islet hormone, acts as a diabetogenic hormone by increasing the hepatic glucose output thereby elevating blood glucose.

Recent studies and tests made by the present inventors have focused on especially the diterpenoid glycoside stevioside which is a major constituent found in the leaves of Stevia rebaudiana where it may occur in amounts of up to about 10% (Hansson J. R., Oliveira B. H., “Stevioside and related sweet diterpenoid glycoside”. Nat. Prod. Rep. 21, p.301-309,1993; Bridel M., Lavielle R., Physiologie Vegetale: “Sur le principe sucre'du Kaa' he'e (Stevia rebaudiana Bertoni): II Les produits d'hydrolyse diastasique du stevioside, glucose et steviol”. Acad. Sci. Paris 192, p. 1123-1125,1931; Soejarto D. D., Kinghorn A. D., Farnsworth N. R., Potential sweetening agent of plant origin. III: “Organoleptic evaluation of Stevia leaf herbarium samples for sweetness”. J. Nat. Prod. 45, p. 590-598,1983; Mossettig E., Nes W. E. Stevioside. II: “The structure of the aglucone”; J. Org. Chem. 20, p. 884-899,1955; Kohda H., Hasai R., Yamasaki K. et al. “New sweet diterpene glucosides from Stevia rebaudiana”. Phytochemistry 15, p. 981-983,1976).

Also, its aglycone, steviol, has been found to be contained in the leaves of Stevia rebaudiana as well as other sweet-tasting glycosides e.g. Steviolbioside, Rebaudioside A, B, C, D and E, and Dulcoside (Bridel M., Lavielle R., Physiologie Vegetale: “Sur le principe sucre'du Kaa' he'e (Stevia rebaudiana Bertoni): II Les produits d'hydrolyse diastasique du stevioside, glucose et steviol”. Acad. Sci. Paris 192, p. 1123-1125,1931; Soejarto D. D., Kinghorn A. D., Farnsworth N. R., Potential sweetening agent of plant origin. III: “Organoleptic evaluation of Stevia leaf herbarium samples for sweetness”. J. Nat. Prod. 45, p. 590-598,1983; Mossettig E., Nes W. E. Stevioside. II: “The structure of the aglucone”; J. Org. Chem. 20, p. 884-899,1955; Mossettig E., Nes W. E. Stevioside. II: “The structure of the aglucone”; J. Org. Chem. 20, p. 884-899,1955; Kohda H., Hasai R., Yamasaki K. et al. “New sweet diterpene glucosides from Stevia rebaudiana”. Phytochemistry 15, p. 981-983,1976).

The inventors of the present invention have successfully proved that both stevioside and steviol have an anti-hyperglycemic, glucagonostatic and insulinotropic effect when administered intravenously to rats and a stimulatory effect on the insulin secretion from mouse islets in vitro.

No well defined, chemical stable, non-toxic, reliable and without adverse effects alternative to the sulphonylureas for the treatment of non-insulin dependent diabetes mellitus is available today and these findings have given rise to further studies and tests of analogues and derivates of these substances in order to find improved and alternative drugs for a self-regulatory treatment of diabetes, hypertension and especially the metabolic syndrome in mammals, preferably man.

In order to prevent sequelae or to delay the development in man of a number of the above-mentioned metabolic and functional disorders it is also aimed to provide new and beneficial dietary supplementations or new self-administrable non-prescription drugs for prophylaxis.

### SUMMARY OF THE INVENTION

According to one aspect of the invention, there is provided a substance or a composition of substances, wherein the substance(s) includes a bicyclo[3.2.1]octan of the structural formula I shown herein or a kaurene structure of the structural formula II shown herein for the preparation of a medicament for the use in the treatment of non-insulin dependent diabetes mellitus.

According to another aspect of the invention, there is provided a substance or a composition of substances, wherein the substance(s) includes a bicyclo[3.2.1]octan of the structural formula I or a kaurene structure of the structural formula II for the preparation of a medicament for the use in the simultaneous treatment of non-insulin dependent diabetes mellitus and hypertension.

According to a third aspect of the invention, there is provided a substance or a composition of substances, wherein the substance(s) include a bicyclo[3.2.1]octan of the structural formula I or a kaurene structure of the structural formula II in combination with at least one soy protein and at least one phytoestrogen for the preparation of a medicament for the use in the treatment of the metabolic syndrome.

According to a forth aspect of the present invention there is provided a composition comprising a substance including a bicyclo[3.2.1]octan of the structural formula I and/or a kaurene structure of the structural formula II and further comprising

(a) a soy protein source, selected from isolated soy protein, soy protein concentrate, or soy flour, said soy protein source providing an amount of soy protein, which is at least 45 weight percent of the total protein content of the composition, said total protein content providing at least 15 percent of the total energy content of the composition,

(b) at least one phytoestrogen compound in an amount of more than 0.10 weight percent of the soy protein content of the composition, and

(c) dietary fibres in an amount of more than 4 weight percent of the total weight of the composition on a dry basis.

In a fifth aspect of the present invention provides a composition comprising a substance including a bicyclo[3.2.1]octan of the structural formula I and/or a kaurene structure of the structural formula II and further comprising

(a) isolated soy protein in an amount of at least 50 weight percent of the total protein content of the composition, said total protein content providing at least 15 percent of the total energy content of the composition,

(b) at least one phytoestrogen compound in an amount of more than 0.10 weight percent of the soy protein content of the composition, and

(c) soybean fibres in an amount of more than 4 weight percent of the total weight of the composition on a dry basis.

In another aspect of the present invention provides a composition comprising a substance including a bicyclo[3.2.1]octan of the structural formula I and/or a kaurene structure of the structural formula II and further comprising

(a) isolated soy protein in an amount of at least 50 weight percent of the total protein content of the composition, said total protein content providing at least 15 percent of the total energy content of the composition,

(b) at least one phytoestrogen compound in an amount of more than 0.10 weight percent of the soy protein content of the composition, and

(c) soy cotyledon fibres in an amount of more than 4 weight percent of the total weight of the composition on a dry basis.

### BRIEF DESCRIPTION OF THE DRAWINGS

The invention is further illustrated by means of the following examples and the accompanying drawing, wherein:

FIG. 1 shows the chemical structure of steviol, isosteviol and stevioside,

FIG. 2a shows the effect of stevioside on blood glucose during i.v. glucose tolerance test in normal Wistar rats,

FIG. 2b shows the effect of stevioside on blood glucose during i.v. glucose tolerance test in GK rats,

FIG. 3a shows the effect of stevioside on glucose-induced release during i.v. glucose tolerance test in normal Wistar rats,

FIG. 3b shows the effect of stevioside on glucose-induced release during i.v. glucose tolerance test in GK rats,

FIG. 4a shows the effect of stevioside on glucose-stimulated insulin secretion from isolated mouse islets,

FIG. 4b shows the effect of steviol on glucose-stimulated insulin secretion from isolated mouse islets,

FIG. 5a shows the effect of an i.v. bolus injection of glucose on plasma glucagon levels during an intravenous glucose tolerance test in GK rats,

FIG. 5b shows the effect of an i.v. bolus injection of glucose and stevioside on plasma glucagon levels during a glucose tolerance test in GK rats,

FIG. 6a shows the systolic blood pressure during 6 weeks treatment of GK rats with stevioside,

FIG. 6b shows the diastolic blood pressure in GK rats treated with stevioside.

FIG. 7a shows the effect of 10⁻³ mmol/l stevioside on the insulin secretion from isolated mouse islets in the presence of glucose ranging between 0 and 16.7 mmol/l,

FIG. 7b shows the effect of 10⁻⁶ mmol/l steviol on the insulin secretion from isolated mouse islets in the presence of glucose ranging between 0 and 16.7 mmol/l,

FIG. 8a-d shows the acute effects of stevioside in type II diabetic patients,

FIGS. 9a-g shows the effects of the action of the combination of stevioside and soy based dietary supplementation in diabetic GK-rats.

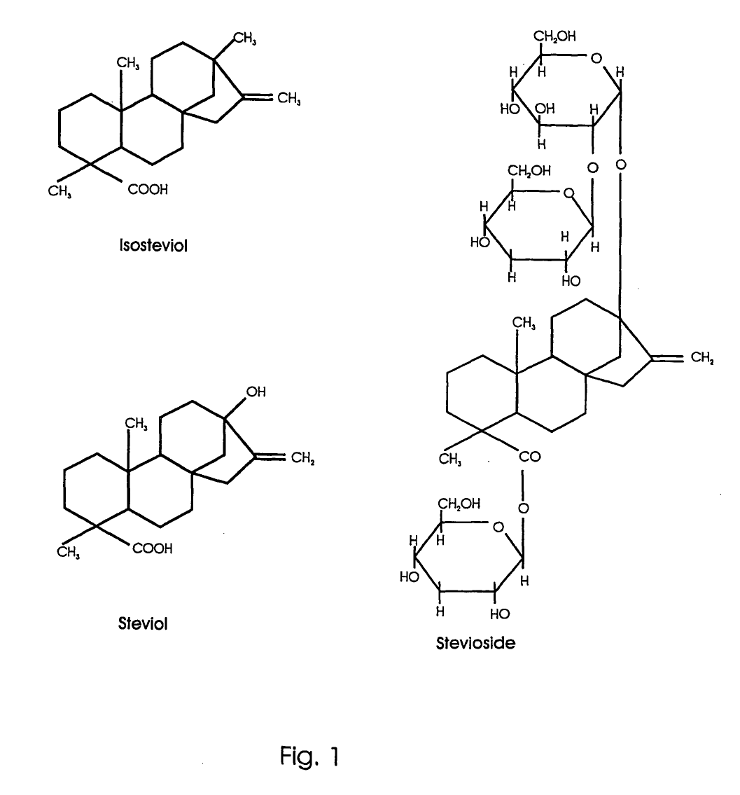

### DETAILED DECRIPTION OF THE PREFERRED EMBODIMENTS

Careful structural chemistry studies by the inventors have revealed that all potential substances for stimulating the insulin secretion extracted from the leaves of Stevia rebaudiana share the common unique skeletal structure of bicyclo[3.2.1]octan of the formula I:

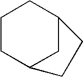

This bicyclo[3.2.1]octan can be found in e.g. steviol, isosteviol and in stevioside. The formula I structure has also been recognised in glucosilsteviol, gymnemic acid, steviolbioside, Rebaudioside A, Rebaudioside B, Rebaudioside C, Rebaudioside D, Rebaudioside E and Dulcoside A.

All these substances also share the common structure of formula II:

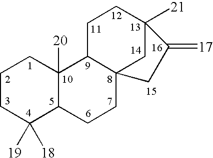

which is the basic structure in kaur-16-en-18-oic acid.

These specific structures of the formula I or II are recognised in several chemical compounds, which have been shown to have a highly potent insulin stimulating effect on isolated mouse pancreatic β-cell, and these structures of formula I and II are evidently the active parts of the molecules in causing the stimulating task.

This assumption is further confirmed by the fact that tests have shown that steviol having the smallest skeletal structure stimulate the insulin secretion to a greater extent than e.g. the glycoside stevioside having a much larger skeletal structure. Also, the inventors of the present invention have succeeded in purifying the different Rebaudiosides from Stevia rebaudiana and preclinical animal studies indicate the same stimulatory effect on insulin secretion.

Consequently this indicate that other compounds including the structures of the formula I or II, such as e.g. analogues, derivates and metabolites of the compounds mentioned above can be used alternatively.

Studies and tests on rats have disclosed that the insulin stimulating effect of these substances is dependent on the concentration of the plasma glucose.

The substances comprising the chemical structures, which includes the formula I or II, did not cause an insulin release as long as the plasma glucose concentration was below approximately 6 mmol/l. At plasma glucose concentration above 6 mmol/l, the stimulating effect of the compounds provided an elevated plasma insulin concentration resulting in an immediate suppression of plasma glucose concentration thereby keeping this at a normal level.

In addition to the above findings, the present inventors have surprisingly found that the substances comprising the chemical structures including the formula I or II also have the capabilities of reducing the glucagon concentration in the blood.

This characteristic nature and qualities of the said substances make them an obvious choice as a component in a medicament for the treatment of especially non-insulin dependent diabetes mellitus (NIDDM).

The finding that e.g. intravenously administered stevioside inhibited blood glucose responses to intravenous glucose in NIDDM rats (GK rats) but not in normal rats supports this fact. This finding is new and surprisingly has neither been expected nor demonstrated in earlier studies which has only been concerned with normal pancreatic islet cells.

As a further example of the unique action of the substances according to the invention, stevioside infusion at normal blood glucose did not cause any hypoglycemia irrespective of it being given as a bolus or at a constant intravenous infusion.

Due to the insulin secretory stimulating effect induced by a slightly elevated plasma glucose concentration, the simultaneous plasma glucagon reducing effect and the inhibited blood glucose response, these substances are able to control, regulate and adjust the plasma glucose concentration of a NIDDM patient to a normal level.

As a consequence of the glucose-dependency the substances only act when needed, e.g. after the patient has increased blood glucose after having eaten. In NIDDM patients treated with medicaments including these substances hypoglycemia will not occur and hypoglycemia will be counteracted.

Therefore, the substances provide a self-regulatory system responding only at elevated plasma glucose concentration.

The treatment with a medicament including these substances provides an attractive alternative to different types of drugs available and presently used today for the treatment of NIDDM, such drugs being drugs for stimulating the insulin secretion (sulphonylureas or repaglinide), drugs for improving the insulin sensivity (biguanides and thiazolidinediones) or drugs for retarding gastrointestinal carbohydrate absorption (α-glucosidase inhibitors).

The potential of these new substances has for the first time also been tested in human NIDDM studies and the beneficial and advantageously combined multiple effects in humans of a single substance according to the invention has been demonstrated and will be described later in the present description and the examples.

The above-mentioned human tests has been conducted by orally administrating the substances, but within the scope of the invention the substances can optionally be used in the preparation of medicaments for intravenous, subcutaneous or intramuscular medication.

The substances further bring along the blood pressure reducing effect. In long-term experiments stevioside suppresses blood pressure in diabetic rat. This important discovery is of the benefit to the diabetic patients that have developed hypertension in relation to or besides their disease.

When at least one of the substances according to the invention is combined in a medicament also comprising at least one soy protein, and at least one phytoestrogen and at least one dietary fibre, it is possible to manufacture a combined preparation of a drug for the treatment of patients with the metabolic syndrome in accordance with the previous definition. Such a medicament may advantageously be used in prophylactic treatment of patient in a risk group. For example, a slow-release drug on the basis composition mentioned above provides a convenient treatment for the patient with the metabolic syndrome.

The inventors of the present invention have demonstrated that the combination of the substances according to the invention and at least one soy protein and at least one phytoestrogen and at least one dietary fibre have a new unexpected and surprisingly synergistic effect surpassing the additive effect of the single components of the medicament thereby providing a completely new and very important medicament for therapeutic or prophylactic treatment of the metabolic syndrome.

The soy protein is preferably the main or sole protein source in a nutritional composition according to the present invention. However, parts of the protein may be provided by other sources such as e.g. skimmed milk, preferably as a powder, and other vegetable or animal proteins including diary proteins. Preferably, at least 45 weight percent, such as 50 weight percent, for example at least 60 weight percent, such as at least 70 weight percent, for example at least 75 weight percent, such as at least 80 weight percent, for example at least 85 weight percent, such as at least 90 weight percent, for example at least 95 weight percent, such as at least 98 weight percent of the total protein content of the composition is soy protein, and more preferably substantially all of the protein is soy protein.

In a preferred embodiment of the invention the soy protein is provided by isolated soy protein. In this embodiment, preferably at least 50 weight percent, for example at least 60 weight percent, such as at least 70 weight percent, for example at least 75 weight percent, such as at least 80 weight percent, for example at least 85 weight percent, such as at least 90 weight percent, for example at least 95 weight percent, such as at least 98 weight percent of the total protein content of the composition is isolated soy protein, and more preferably substantially all of the protein is provided by isolated soy protein.

The total protein content of a composition according to the present invention provides at least 15 percent of the total energy content of the composition, for example 18 percent, such as at least 20 percent, for example at least 22 percent, such as at least 25 percent, for example at least 28 percent, such as at least 30 percent, for example at least 32 percent, such as at least 35 percent, for example at least 38 percent, such as at least 40 percent, for example at least 42 percent, such as at least 45 percent, for example at least 48 percent, such as at least 50 percent of the total energy content of the composition, and preferably less than 90 percent of the total energy content of the composition.

Phytoestrogen compounds according to the present invention are defined as naturally occurring plant substances, said substances being either structurally or functionally similar to 17β-estradiol or generating estrogenic effects. Phytoestrogens consist of a number of classes including isoflavones, coumestans, lignans and resorcylic acid lactones. Examples of isoflavones according to the present invention are genistein, daidzein, equol, glycitein, biochanin A, formononetin, and O-desmethylangolesin. The phytoestrogen compounds of a composition according to the present invention are preferably isoflavones, more preferably genistein, daidzein, glycitein and/or equol, yet more preferably genistein and/or daidzein, and even more preferably genistein. A preferred composition according to the present invention may accordingly comprise a single isoflavone, such as genistein, daidzein, glycitein or equol, or it may comprise at least one isoflavone selected from the group consisting of at least genistein, daidzein, glycitein and equol. When present in the plant the isoflavones are mainly in a glucoside form, i.e. attached to a sugar molecule. This glucoside form can be deconjugated to yield a so-called aglycone form, which is the biologically active species. A composition according to the present invention may comprise isoflavones in glucoside and/or aglycone forms regardless of whether the deconjugation to the aglycone form has taken place biologically, in vitro or by any other means whereby the isoflavones are included in a composition according to the present invention or if the aglycone forms are the native form of the isoflavones.

The phytoestrogen compound is preferably present in an amount of at least about 0.12 weight percent of the soy protein content, such as at least about 0.14 weight percent, for example at least about 0.16 weight percent, such as at least about 0.18 weight percent, for example at least about 0.20 weight percent, such as at least about 0.22 weight percent, for example at least about 0.24 weight percent, such as at least about 0.25 weight percent, for example more than about 0.25 weight percent, such as at least about 0.26 weight percent, for example at least about 0.28 weight percent, such as at least about 0.30 weight percent, for example at least about 0.32 weight percent, such as at least about 0.33 weight percent, for example more than about 0.33 weight percent, such as at least about 0.35 weight percent, for example at least about 0.40 weight percent, such as at least about 0.45 weight percent, for example at least about 0.50 weight percent, such as at least about 0.55 weight percent, for example at least about 0.60 weight percent, such as at least about 0.65 weight percent, for example at least about 0.70 weight percent, such as at least about 0.75 weight percent, for example at least about 0.80 weight percent, such as at least about 0.85 weight percent, for example at least about 0.90 weight percent, such as at least about 1.0 weight percent of the soy protein content, and preferably less than 2.50 weight percent of the soy protein content.

In the past, the downstream processing techniques used in the preparation of soy proteins have included steps that removed and/or destroyed isoflavones. Methods are available today, which provide soy protein products with high, fixed levels of naturally occurring isoflavones. The isoflavones according to the present invention in glucoside and/or aglycone forms can be included in a composition according to the present invention as part of such soy protein products and/or by themselves and/or as part of any other composition comprising isoflavones.

The dietary fibres used in the present invention should preferably comprise a mixture of insoluble fibres and water-soluble fibres also referred to as soluble fibres. Soluble fibres have a lowering effect on blood cholesterol levels. Examples of dietary fibres comprising soluble fibres are fibres from apples, bananas, oranges, carrots, oats, and soybeans. The dietary fibres preferably comprise soluble fibres in an amount of about 5 weight percent, such as about 10 weight percent, for example about 15 weight percent, such as about 20 weight percent, for example about 25 weight percent, such as about 30 weight percent, for example about 35 weight percent, such as about 40 weight percent, for example about 45 weight percent, such as about 50 weight percent, for example about 55 weight percent, such as about 60 weight percent, for example about 65 weight percent, such as about 70 weight percent, for example about 75 weight percent, such as about 80 weight percent, for example about 85 weight percent, such as about 90 weight percent, for example about 95 weight percent. The dietary fibres used in the present invention are preferably soybean fibres, more preferably soy cotyledon fibres. Such fibres are derived from dehulled and defatted soybean cotyledon and are comprised of a mixture of soluble and insoluble fibres. Soy cotyledon fibres are distinctly different from soybean fibres derived from soy hulls as well as other fibre sources. Soy cotyledon fibres are bland tasting, contain no cholesterol, are low in fat and sodium, and they have good water-binding properties and low caloric content.

The amount of dietary fibres of the total weight of a composition according to the present invention on a dry basis is preferably more than 4 weight percent, for example at least 5 weight percent, such as at least 6 weight percent, for example at least 7 weight percent, such as at least 8 weight percent, for example at least 9 weight percent, such as at least 10 weight percent, for example at least 11 weight percent, such as at least 12 weight percent, for example at least 13 weight percent, such as at least 14 weight percent, for example at least 15 weight percent, such as at least 16 weight percent, for example at least 17 weight percent, such as at least 18 weight percent, for example at least 19 weight percent, such as at least 20 weight percent, and preferably less than 50 weight percent.

Preferred amounts of dietary fibres as a weight percent of the content of soy protein, shall be in the range of from about 10 to 100 weight percent, and preferred amounts are in the range of from 15 to 90 weight percent, such as from 20 to 80 weight percent, for example 25 weight percent, such as 30 weight percent, for example 33 weight percent, such as 35 weight percent, for example 40 weight percent, such as 50 weight percent, for example 60 weight percent, such as 70 weight percent, for example 75 weight percent.

Accordingly, the weight ratio of soy protein to dietary fibres is from about 1.0 to about 10.0, preferably more than about 1.0, for example about 1.25, such as at least about 1.5, for example at least about 1.75, such as at least about 2.0, for example at least about 2.25, such as at least about 2.5, for example at least about 2.75, such as at least about 3.0, for example at least about 3.25, such as at least about 3.5, for example at least about 3.75, such as at least about 4.0, for example at least about 4.25, such as at least about 4.5, for example at least about 4.75, such as at least about 5.0, for example at least about 5.5, such as at least about 6.0, for example at least about 7.5.

The preferred daily dosage of soybean fibres is from at least 1 g to about 100 g soybean fibres, for example from at least 2 to about 75 g soybean fibres, such as from at least 3 g to about 50 g, for example from at least 4 g to about 40 g, such as from at least 5 to about 30 g, such as from at least 10 g to about 20 g soybean fibres.

Alternatively, the present invention provides a composition wherein no soy protein is present and wherein the dietary fibres are soy cotyledon fibres. This composition comprises soy cotyledon fibres in an amount of more than 4 weight percent of the total weight of the composition on a dry basis, and at least one phytoestrogen compound in an amount of more than 0.10 weight percent of the soy cotyledon fibres of the composition. The present invention also provides the use of such a composition as a medicament and/or in the manufacture of a medicament effective in treating and/or alleviating type 2 diabetes, the metabolic syndrome or a cardiovascular disease associated therewith in subjects with diabetes and/or the metabolic syndrome. The present invention also provides the use of such a composition and/or such a composition for use in treating type 2 diabetes, the metabolic syndrome or related cardiovascular diseases in a subject.

When no soy protein is present in the composition, the phytoestrogen compound is preferably present in an amount of at least about 0.12 weight percent of the soy cotyledon fibre content, such as at least about 0.14 weight percent, for example at least about 0.16 weight percent, such as at least about 0.18 weight percent, for example at least about 0.20 weight percent, such as at least about 0.22 weight percent, for example at least about 0.24 weight percent, such as at least about 0.25 weight percent, for example more than about 0.25 weight percent, such as at least about 0.26 weight percent, for example at least about 0.28 weight percent, such as at least about 0.30 weight percent, for example at least about 0.32 weight percent, such as at least about 0.33 weight percent, for example more than about 0.33 weight percent, such as at least about 0.35 weight percent, for example at least about 0.40 weight percent, such as at least about 0.45 weight percent, for example at least about 0.50 weight percent, such as at least about 0.55 weight percent, for example at least about 0.60 weight percent, such as at least about 0.65 weight percent, for example at least about 0.70 weight percent, such as at least about 0.75 weight percent, for example at least about 0.80 weight percent, such as at least about 0.85 weight percent, for example at least about 0.90 weight percent, such as at least about 1.00 weight percent, for example at least about 1.25 weight percent, such as at least about 1.50 weight percent, for example at least about 1.75 weight percent, such as at least about 2.00 weight percent, for example at least about 2.50 weight percent, such as at least about 3.00 weight percent, for example at least about 3.5 weight percent, such as at least about 5.00 weight percent of the soy cotyledon fibre content of the composition, and preferably less than 10.00 weight percent of the soy cotyledon fibre content of the composition.

Alternatively, the present invention provides a composition wherein no dietary fibres are present. This composition comprises soy protein, preferably isolated soy protein in an amount of at least 50 weight percent of the total protein content of the composition, said total protein content providing at least 15 percent of the total energy content of the composition, and at least one phytoestrogen compound in an amount of more than 0.10 weight percent of the soy protein content of the composition. The present invention also provides the use of such a composition in the treatment of diabetic subjects, said treatment being particularly effective in lowering serum levels of glucose and lipids in a subject. The present invention also provides the use of such a composition comprising soy protein and a phytoestrogen compound as a medicament and/or in the manufacture of a medicament for treating diabetic subjects, said treatment being effective in lowering serum levels of glucose and/or insulin and/or lipids. The present invention also provides the use of such a composition as a medicament and/or in the manufacture of a medicament effective in treating and/or alleviating type 2 diabetes, the metabolic syndrome as defined herein and/or any cardiovascular disease associated therewith in a subject.

A composition according to the present invention may optionally comprise a carbohydrate source, a fat source, flavouring agents, vitamins, minerals, electrolytes, trace elements and other conventional additives. The nutritional composition according to the present invention may in one embodiment also comprise one or more flavouring agents such as cocoa, vanilla, lime, strawberry or soup flavours, such as mushroom, tomato or bouillon, and/or and sweeteners such as aspartame as well as other additives such as xanthan gum.

When a carbohydrate source is present in a composition according to the present invention, it is preferably present in an amount of less than 30 weight percent such as less than 25 weight percent of the composition. Preferably, the amount of carbohydrate amounts to at least 5 weight percent, more preferred at least 10 weight percent, and most preferred at least 15 weight percent, of the composition. Lecithinated fat reduced cacao is particularly preferred. Other preferred carbohydrates for use in a composition according to the present invention are polydextrose or saccharose, but these should be limited using other sweeteners like e.g. aspartame.

When a fat source is present in a composition according to the present invention, it is usually present in an amount from 0.5 to 10 weight percent, preferably 1 to 9 weight percent, such as from 1.5 to 8 weight percent, for example from 2 to 7 weight percent, such as from 2.5 to 6 weight percent of the composition. The fat source will preferably comprise polyunsaturated fatty acids and monounsaturated fatty acids and optionally also saturated fatty acids. Soy lecithins and α-linolenic acids are particularly preferred. The amount of polyunsaturated fatty acids and monounsaturated fatty acids, including the essential fatty acids, may range from 35 to 50, preferably 38 to 44, weight percent of the total amount of the fat source. The essential fatty acids are also called omega-6 and omega-3 fatty acids and include linolic acid and/or linolenic acid (α-linolenic acid). The amount of saturated fatty acids may be from 20 to 30 weight percent, preferably 22 to 26 weight percent, of the total amount of fat.

Vitamins and minerals may optionally be added to a composition according to the present invention in accordance with the limits laid down by health authorities. The vitamins will typically include A, B1, B2, B12, folic acid, niacin, panthotenic acid, biotin, C, D, E and K. The minerals will typically include iron, zinc, iodine, copper, manganese, chromium and selenium. Electrolytes, such as sodium, potassium and chlorides, trace elements and other conventional additives may also be added in recommended amounts.

According to one presently preferred hypothesis, a composition according to the present invention will alleviate abnormalities associated with apolipoprotein and lipoprotein particle distribution and promote a decreased plasma VLDL and remnant level, a decrease in the apoE concentration in VLDL and remnants, a decrease in the amount of small dense LDL, and a HDL particle distribution similar to that of a comparable non-diabetic, healthy individual.

Hypertriglyceridaemia in diabetes is associated with an increase in the clotting activities of thrombogenic factors such as factor VII and factor X and an increase in the level of the inhibitor of tissue plasminogen activator, PAI-1. The increased inhibitor concentration results in a decreased level of plasminogen synthesis and thus a decreased level of plasminogen stimulated clot lysis. These changes in clotting activities no doubt contribute to the observed procoagulant state in diabetes. Accordingly, the present invention provides a composition, which may be effective in normalising the clotting activities of thrombogenic factors such as factor VII and factor X by e.g. decreasing the increased activity thereof observed in a subject diagnosed as having type 2 diabetes or diagnosed as having an impaired glucose tolerance or a decreased insulin sensitivity. Also, a composition according to the present invention may be effective in promoting a decrease in the concentration of the inhibitor of tissue plasminogen activator, PAI-1, which in turn leads to an increased plasminogen stimulated clot lysis. A composition according to the present invention may also be effective in reducing an increased platelet aggregatability and/or mediating directly or indirectly a reduction of the increased level of lipoprotein (a) associated with a procoagulant state in a diabetic condition.

Hyperinsulinaemia is also considered a risk factor for coronary heart disease (CHD) in diabetic subjects due to the association of high insulin levels with increased incidence and mortality rates of CHD. A composition according to the present invention may be effective in lowering serum insulin levels in subjects diagnosed with type 2 diabetes. Diabetic patients having increased endogenous insulin levels, i.e. subjects diagnosed with type 2 diabetes, or having increased peripheral circulating insulin levels as a result of intermittent injections of large amounts of exogenous insulin are particularly prone to hyperinsulinaemia.

In one embodiment the present invention provides a composition effective in reducing and/or eliminating risk factors for coronary heart disease (CHD) in obese subjects suffering from a diabetic condition and/or the metabolic syndrome. Consequently, a composition according to the present invention may be capable of preventing, alleviating, treating and/or eliminating hyperinsulinaemia and/or hyperglycaemia and/or hypertension and/or hypertriglyceridaemia and/or hypercholesterolaemia and/or effective in mediating an increase in the low serum levels of HDL-cholesterol.

It is very possible that type 2 diabetes is also associated with insulin resistance and hyperinsulinaemia independently of an increase in abdominal lipids. Hyperinsulinaemia in turn is associated with dyslipidaemia, i.e. increased VLDL, decreased and altered HDL and increased small dense LDL, and with hypertension, all of which are risk factors for atheriosclerosis. This array of abnormalities and disorders, or a part of thereof, is generally termed the insulin resistance syndrome, or syndrome X, or metabolic syndrome.

In one embodiment, a composition according to the present invention may be capable of effectively decreasing and/or eliminating increased serum levels of VLDL and/or LDL, and/or increasing decreased serum levels of HDL, and of decreasing and/or eliminating serum LDL levels including serum levels of small dense LDL. A composition according to the present invention may also be capable of reducing an elevated level of small, dense LDL-particles and/or reducing an elevated ratio of LDL-apoB to LDL-cholesterol and/or preventing, treating or alleviating hypertension.

Since insulin can be expected to be capable, either in combination with other compounds such as additional growth factors, or on its own, of increasing the levels of intracellular cholesterol, by e.g. increasing a delivery of LDL-cholesterol via the LDL-receptor, and concomitantly therewith increase an endogenous biosynthesis of cholesterol that makes yet more cholesterol available for new membrane synthesis in the cell proliferation process, it is an object of the present invention to counteract any increased activity including any insulin stimulated increased activity of the LDL-receptor.

It is also possible that insulin and other growth factors have the potential to promote the accumulation of cholesterol intracellularly. This may in fact well occur in a diabetic subject and more generally under conditions when cells are stimulated, but cannot proliferate normally. Accordingly, a composition of the present invention may also be capable of alleviating, eliminating and/or treating any decrease, including any insulin mediated decrease, in the HDL receptor-mediated cholesterol efflux. Accordingly, a composition according to the present invention may be capable of reducing and/or eliminating any enhanced retention of intracellular cholesterol caused by a decreasing HDL receptor-mediated cholesterol efflux.

Modifications to lipoproteins are another risk factor for cardiovascular disease in diabetes. The modification characterised by protein glycosylation is associated with diabetes, and glycosylated lipoproteins such as e.g. LDL, IDL, VLDL and HDL can be expected to be functionally abnormal. Accordingly, the accumulation of glycosylated LDL in the plasma of a diabetic subject can be perceived to enhance cholesterol ester accumulation. Also, glycosylation of HDL can be expected to impair the ability of HDL binding to the HDL receptor. This impaired binding is likely to reduce the level of intracellular cholesterol efflux. Accordingly, glycosylated HDL may well be another factor potentially contributing to the accumulation of cholesterol in the arterial cell wall. A composition according to the present invention may be effective in preventing, alleviating, treating, reducing and/or eliminating lipoprotein glycosylation in a diabetic subject. In addition, a composition according to the present invention may also be effective in preventing lipoprotein modification caused e.g. by oxidation, chemical modification such as chemical cross-linking, or modifications caused by an alteration in the lipid composition of the lipoprotein, such as any increase or decrease in the content of triglycerides, cholesterol esters, free cholesterol, and apolipoproteins.

Glycosylated lipoproteins have been suggested to be the subject of further processing leading to the formation of hyperglycosylated compounds. Glycosylation and hyperglycosylation of proteins including lipoproteins in both plasma and the arterial wall can also be expected to be a risk factor for cardiovascular disease including arteriosclerosis in diabetic subjects. Accordingly, a composition according to the present invention may be capable of preventing, treating, reducing, alleviating and/or eliminating the accumulation of hyperglycosylated proteins in both serum and cells of the arterial wall. By doing so, the composition is acting to decrease the amount of LDL becoming “trapped” in the arterial wall due to the high degree of glycosylation of arterial wall proteins. A composition according to the present invention may also be effective in alleviating and/or preventing any change to the endothelial cell wall that increase LDL “trapping”, and it may be effective in restoring the formation of cells with normal permeability and adhesion parameters.

Lipoprotein glycosylation, hyperglycosylation, oxidation and/or auto-oxidative glycosylation, are risk factors for cardiovascular disease such as arteriosclerosis in diabetes. Accordingly, a composition according to the present invention may be effective in eliminating, preventing, alleviating, treating and/or reducing any incidence of lipoprotein glycosylation, hyperglycosylation, oxidation and/or auto-oxidative glycosylation. According to one presently preferred hypothesis, the phytoestrogen compound of a composition according to the present invention is capable of counteracting incidences. The phytoestrogen compound may also be capable of preventing, reducing and/or eliminating the formation of e.g. free radicals that are likely to be involved in such processes, and a composition according to the present invention may be effective in being, promoting, and/or facilitating the formation of an effective antioxidant defence system for counteracting glycosylation, hyperglycosylation, oxidation and/or auto-oxidative glycosylation of serum proteins and proteins including lipoproteins of the arterial cell wall.

Since oxidative stress is a characteristic of diabetes and possibly a contributory factor to among others lipoprotein oxidation and/or glycosylation, and since no efficient antioxidant protection exists due to e.g. significantly decreased levels in diabetic subjects of antioxidants such as e.g. ascorbic acid, a composition according to the present invention may be effectively acting as an antioxidant in preventing lipoprotein oxidation and/or glycosylation.

A composition according to the present invention may be effectively acting as an antioxidant in preventing lipoprotein oxidation and/or glycosylation. By the term auto-oxidative glycosylation, or glycoxidation, is understood a reaction catalysed e.g. by reducing sugars that leads to an oxidative modification and/or cross-linking of proteins. The rate of such a process can be expected to be increased in the presence of high glucose concentrations since the oxidising potential is significantly increased under such circumstances. An increased production of free radicals and lipid peroxidation may also contribute to the formation of auto-oxidative glycosylated lipoproteins and this contribution may also be effectively prevented and/or eliminated by a composition according to the present invention.

According to another presently preferred hypothesis, the binding of a phytoestrogen compound, such as e.g. isoflavones, optionally in combination with soy peptides preferably provided by hydrolysis of soy protein, to a receptor in the arterial wall, such as e.g. the estrogen receptor, or an estrogen-like receptor, is involved in or effective in controlling uptake of cholesterol and/or triglycerides in the arterial wall, possibly by regulating the permeability of said wall and/or the mechanism of cholesterol and/or triglyceride transport across cellular membranes. Consequently, the binding of isoflavones such as e.g. genistein and/or daidzein to a receptor in the arterial wall may prevent cholesterol and/or triglycerides from entering the arterial wall, or reduce and/or substantially eliminate the amount of cholesterol and/or triglycerides that enters the arterial wall. Receptor binding of isoflavones in the arterial wall is particularly effective in controlling, preventing and/or eliminating fatty streak formation and/or fibrous plaque development and/or effective in mediating a regression of one or both of said arteriosclerotic conditions.

According to a particularly preferred hypothesis, binding of isoflavones such as e.g. genistein and/or daidzein to a receptor in the arterial wall, preferably an estrogen receptor or an estrogen-like receptor, results in an increased nitric oxide synthesis in the endothelial cells of the arterial wall. Nitric oxide is known to exert anti-arteriosclerotic effects including inhibition of platelet adhesion and aggregation, and of smooth muscle cell proliferation. Soy peptides obtainable by hydrolysis of soy protein may participate in the binding of isoflavones to an estrogen receptor or an estrogen-like receptor or the soy peptides may themselves bind to said receptor and exert an action leading to an increased nitric oxide synthesis.

In one presently preferred hypothesis, the establishment of an oxidative potential occurs concomitantly with, and is very likely caused by, a decrease in cellular antioxidant defence systems. This hypothesis is supported by the fact that e.g. ascorbic acid concentrations are decreased in many diabetic individuals. Accordingly, a composition according to the present invention may be effective in acting as an antioxidant. This action reduces and/or eliminates LDL, VLDL, IDL and/or HDL susceptibility to oxidation. Concomitantly with a direct anti-oxidative effect, a composition according to the present invention may also lower the increased serum glucose levels and by doing so, a composition according to the present invention may be effective in reducing the oxidising potential causing and/or contributing to oxidative stress.

Furthermore, a composition according to the present invention may also be effective in reducing an enhanced susceptibility to endothelial injury and/or for alleviating and/or restoring and/or improving an inefficient endothelial cell repair mechanism. One effect of such an action exerted by a composition according to the present invention is to direct macrophage development away from foam cell formation and to increase the potential of generating arterial smooth muscle cells.

The unique dyslipidaemia associated with type 2 diabetes is a major risk factor for cardiovascular disease, and prevention, alleviation, reduction and/or elimination of dyslipidaemia in diabetic subjects is a prime objective of administration of a composition according to the present invention to a diabetic individual. Another important objective of such an administration is the development in a diabetic subject of a gradually reduced insulin resistance and/or a gradually improved glucose tolerance. Since increasing insulin resistance and impaired glucose tolerance are key elements in the progression of type 2 diabetes, the same factors most also be a natural focus of any preventive treatment.

In another presently preferred hypothesis, a composition according to the present invention will promote and/or mediate a reduction in arterial wall thickness and lead to a reduction in the amount of LDL entering the wall. It is believed that an increased thickness of the arterial wall is positively associated with an increased uptake of LDL-particles that are likely to either aggregate or oxidize within the cells of the arterial wall.

Also, a composition according to the present invention may be capable of reducing, eliminating and/or preventing the formation of increased serum levels of lipoprotein (a) in a diabetic subject. Lipoprotein (a) levels may primarily be genetically determined, and no current cardiovascular medications are thought effective in lowering serum levels of lipoprotein (a).

Without wishing to be bound by any specific theory it is presently believed that both soluble dietary fibres (working as nutrients) and insoluble dietary fibres (working as bulking agents), in particular from soybean fibres, more particularly soy cotyledon fibres, provide favourable growth conditions for the microflora in the human gut, which makes the microflora more effective in deconjugating isoflavones in the glucoside form to the aglycone form. Isoflavones in the aglycone form are absorbed faster and to a greater extent in the human gut than isoflavones in the glucoside form, and isoflavones in the aglycone form are the biologically more active species. In view hereof it can be understood that administration of a combination of soy proteins, a high, fixed level of isoflavones and a combination of soluble and insoluble fibres is effective in providing an increased uptake of isoflavones.

A composition according to the present invention may be used as a food for special dietary use, preferably for lowering serum levels of glucose and/or for lowering serum levels of insulin and/or for lowering total serum cholesterol and/or LDL-cholesterol and/or triglyceride levels and/or for lowering blood pressure and/or for increasing glucose tolerance and/or insulin sensitivity and/or for preventing and/or alleviating and/or treating impaired glucose tolerance and/or insulin secretory failure in diabetic subjects and/or for preventing and/or alleviating and/or treating an arteriosclerotic condition by reducing the influx of lipoproteins and/or cholesterol and/or triglycerides into the endocelium of the arterial wall of a diabetic subject suffering from a cardiovascular disease. For example, from one to three daily meals of ordinary food can be supplemented or replaced by a composition according to the present invention. Hereby, significant reductions in serum levels of total cholesterol and LDL-cholesterol and triglyceride can be obtained, as well as an improvement of HDL/LDL-cholesterol ratio and/or an increase in serum HDL-cholesterol levels. The composition may provide from 50 to 250 kcal per serving.

The present invention also provides a composition according to the invention in the form of a micronutrient. In this connection a micronutrient is a nutritional supplement and/or a pharmacological composition and/or a medicament comprising i) a synthetic phytoestrogen-like compound capable of binding to an estrogen receptor or an estrogen-like receptor, and/or ii) a naturally occurring, plant-extractable compound in an amount, on a weight per weight basis, in excess of the amount of said compound, when it is present in a natural host such as a plant cell from which the compound can be extracted or isolated, and optionally iii) soy peptides obtainable from a partial hydrolysis of soy protein.

The naturally occurring, plant-extractable compound is preferably but not limited to compounds capable of binding to an estrogen receptor, an estrogen-like receptor, a beta-2-adrenergic receptor or a receptor belonging to the class of beta-2-adrenergic receptors. When the naturally occurring compounds are isolated from plants such as soybeans, they may be selected from the group at least containing phytoestrogens such as soybean phytoestrogens such as soybean isoflavones, soy protein or fragments thereof, e.g. peptides or amino acid sequences, soybean fibres, lecithin, linolenic acid, an antioxidant, a saponin, a lignan, a protease inhibitor, a trypsin inhibitor, and a tyrosine kinase inhibitor. Additional constituents of the micronutrient may preferably be selected among a DNA topoisomerase inhibitor, a ribosome kinase inhibitor, a growth control factor such as e.g. epidermal growth factor, transforming growth factor alpha, platelet derived growth factor, and preferably any growth control factor controllable by a tyrosine kinase activity. The micronutrient may also comprise ormeloxifene and/or levormeloxifene as described by among others Holm et al. (1997) in Arteriosclerosis, Thrombosis, and Vascular Biology 17 (10), 2264-2272, and in Clinical Investigation 100 (4), 821-828. When the naturally occurring, compound is an isoflavone, the isoflavone may have been deconjugated to the aglycone form either biologically or in vitro prior to the incorporation in the micronutrient.

In one particularly preferred embodiment the present invention provides a composition or a micronutrient according to the present invention in combination with a functional food ingredient comprising a sterol, preferably an ingredient selected from the group consisting of a stanol ester, a tocotrienol, a mevinolin, and a phytosterol compound such as e.g. campesterol, sitosterol or stigmasterol, or a combination thereof.

According to one preferred embodiment, a composition or a micronutrient according to the present invention is for use as a functional food ingredient. A composition or a micronutrient according to the present invention may also be administered as a probe or by intravenous administration, or in tablet or capsule form. The present invention also provides a pharmaceutical preparation comprising a composition or a micronutrient according to the present invention, use of the a composition or a micronutrient according to the present invention in therapy and/or a diagnostic method performed on the human or animal body, use of a composition or a micronutrient according to the present invention in the manufacture of a medicament and use of a composition or a micronutrient according to the present invention in the manufacture of a medicament for treating a subject suffering from type 2 diabetes, the metabolic syndrome or cardiovascular diseases associated therewith.

The micronutrient is particularly useful in the prevention and/or treatment of type 2 diabetes, the metabolic syndrome and cardiovascular diseases associated therewith in a diabetic subject.

In one embodiment the present invention provides a composition according to the present invention for use as a medicament or as a dietary preparation. A composition according to the present invention for use as a medicament or as a dietary preparation may be used in preventing, alleviating, eliminating and/or treating type 2 diabetes and/or a cardiovascular disease associated therewith. The present invention also provides the use of a composition according to the present invention for the manufacture of a medicament for preventing, alleviating and/or treating type 2 diabetes and/or a cardiovascular disease in a diabetic subject.

A composition according to the present invention for use as a medicament and/or the use of a composition according to the present invention for the manufacture of a medicament for treating a subject with diabetes and/or the metabolic syndrome and/or a cardiovascular disease associated therewith may be effective in i) lowering serum glucose levels and/or ii) reducing the influx of cholesterol and/or triglycerides into the arterial wall and/or the amount of oxidized LDL-cholesterol present in the arterial wall and/or iii) lowering total serum cholesterol and/or serum LDL-cholesterol and/or serum triglyceride levels and/or serum homocystein levels and/or lowering blood pressure and/or increasing the HDL/LDL-cholesterol ratio and/or serum HDL-cholesterol levels and/or iv) increasing glucose tolerance and/or insulin sensitivity and/or v) alleviating impaired glucose tolerance and/or insulin secretory failure and/or vi) preventing, alleviating, eliminating and/or treating cardiovascular diseases, such as e.g. hypertriglyceridaemia, hypercholesterolaemia, arteriosclerosis, atherosclerosis, arteriolosclerosis, angina pectoris, thrombosis, myocardial infarction, hypertension, hyperglycaemia, and hyperinsulinaemia, in a diabetic subject.

A composition according to the present invention for use as a medicament and/or the use of a composition according to the present invention for the manufacture of a medicament may also be effective in treating cardiovascular diseases such as e.g. fatty streak formation and/or fibrous plaque development and/or complicated lesion development. Furthermore, a composition according to the present invention for use as a medicament and/or the use of a composition according to the present invention for the manufacture of a medicament may also be effective in treating a procoagulant state and/or an increased activity of clotting factors, insulin resistance, glycosidation and/or oxidation and/or chemical modification of lipoproteins, as well as impaired glucose tolerance.

The present invention also provides a method of preventing and/or treating by therapy type 2 diabetes and/or the metabolic syndrome in a human or animal body, said method comprising administration to said human or animal body of a composition according to the present invention in an amount effective in lowering serum glucose levels and/or reducing the influx of cholesterol and/or triglycerides into the arterial wall and/or reducing the amount of oxidized LDL-cholesterol present in the arterial wall and/or lowering serum cholesterol levels and/or lowering LDL-cholesterol levels and/or lowering serum triglyceride levels and/or serum homocystein levels and/or lowering blood pressure and/or improving glucose tolerance and/or increasing insulin sensitivity and/or alleviating impaired glucose tolerance and/or improving insulin secretion and/or reducing or eliminating fatty streak formation and/or preventing, reducing or eliminating fibrous plaque formation and/or preventing, reducing or eliminating complicated lesion formation and/or preventing, reducing or eliminating the risk of a subject contracting angina pectoris and/or preventing, reducing or eliminating the risk of a subject contracting a myocardial infarction and/or preventing, treating, prophylactically treating, alleviating and/or eliminating hypertension and/or hyperglycaemia and/or hyperinsulinaemia and/or hypercholesterolaemia and/or hypertriglyceridaemia and/or arteriosclerosis and/or atherosclerosis and/or arteriolosclerosis in a diabetic subject.

The period of treatment is preferably in the range of from 1 to 12 months or more, such as from 2 weeks to 9 months, for example from 3 weeks to 6 months, such as from 4 weeks to 4 months, such as from 6 weeks to 3 months. However, the period of treatment shall not be limited to these periods and may e.g. be longer than 12 months, such as e.g. a lifelong treatment in order to prevent and/or alleviate type 2 diabetes and/or a cardiovascular disease in connection therewith.

In one embodiment the present invention provides a pharmaceutical preparation comprising a composition according to the present invention. The pharmaceutical preparation can be prepared in any way known to the skilled person.

In another embodiment the present invention provides the use of a composition according to the present invention in the manufacture of a nutritional preparation for lowering serum glucose levels and/or serum cholesterol levels and/or serum LDL-cholesterol levels and/or serum triglyceride levels and/or serum homocystein levels and/or lowering blood pressure and/or for increasing the HDL/LDL-cholesterol ratio and/or serum HDL-cholesterol levels in a diabetic subject. The nutritional preparation may take any form, which is suitable for human or animal consumption. In one preferred embodiment, the composition is a powdery mixture, which is suspendable, dispersible or emulsifiable in a liquid for human or animal consumption. The liquid is preferably a water-containing liquid such as e.g. water, coffee, tea or juice. For such a purpose, the composition may be packed in a package intended for covering part of or the total nutritional requirement for a defined period of time, such as a period of e.g. three days or a week. The present invention also provides the nutritional preparation in the form of a dietary supplement.

The nutritional preparation in one embodiment of the present invention is preferably a functional food or drink, i.e. a readily obtainable edible or drinkable substance that is supplemented with a composition according to the present invention to provide a medical or pharmaceutical effect. Accordingly, the present invention provides a composition according to the present invention for use as a functional food ingredient. Functional foods and drinks are preferably selected from the group consisting of diary products, such as yoghurt and yoghurt ice cream, juice, such as orange juice or tomato juice, ready made liquids for drinking, a spreadable product such as e.g. a margarine or a vegetable or plant extracted oil, a cereal product, such as a traditional breakfast cereal product, nutritional bars, biscuits, bread, soups, such as tomato soup, a meat product, such as a hamburger, a meat substitute product, and a vegetable product. In a further embodiment, a nutritional preparation according to the present invention may be in the form of a ready made liquid or in a powder form or in the form of a troche, a solid composition such as a nutritional bar, a fruit bar, a cookie, a cake, a bread or a muffin.

In another embodiment, a composition according to the present invention is a liquid nutritional preparation in a water-containing liquid, in which the solid ingredients are suspended, dispersed or emulgated in an amount of from 10 to 25 weight percent. When the liquid nutritional preparation is intended for drinking, it will usually comprise a flavouring agent as discussed above. However, the liquid nutritional preparation may also be used for probe administration.

In another embodiment, the present invention relates to the use of a composition according to the present invention as a partial or total diet for an overweight subject or an overweight subject suffering from a diabetic condition. Obesity is believed to be one of the major causes of diabetes including type 2 diabetes. Overweight diabetic subjects often have an increased serum cholesterol level and an increased triglyceride level and are therefore more likely to develop cardiovascular diseases. However, the present invention is not limited to treating obese diabetic subjects with an increased risk of contracting a cardiovascular disease, i.e. obese diabetic subjects likely to have increased serum levels of cholesterol and/or triglycerides. A composition according to the present invention also has substantial serum cholesterol, serum LDL-cholesterol and serum triglyceride lowering effect in diabetic subjects that do not also suffer from overweight.

The inventors of the present invention has used the combination of the substances according to the invention and at least one soy protein as a dietary supplementation in human studies. The test results significantly proved, as will be seen in the following examples, that such combination has a beneficial impact on cardiovascular risk markers in type II diabetic subjects.

Furthermore, as will be evident from the presented examples, compositions comprising substances according to the present invention in combination with soy protein, soy isoflavones and soy fibres in specified ratios, have new unexpected and surprising synergistic effects surpassing the additive effects of the individual constituents. Thereby the present invention provides compositions useful in the therapeutic and/or prophylactic treatment of various diseases including the metabolic syndrome.

Stevioside at a dose as high as 15 g/kg body weight was not lethal to either mice, rats or hamsters (Toskulkao C., Chaturat L., Temcharoen P., Glinsukon T. “Acute toxicity of stevioside, a natural sweetener, and its metabolite, steviol, in several animal species”. Drug Chem. Toxicol. 1997 February-May;20(1-2), p. 31-44). In rats and mice, LD₅₀ values of steviol were higher than 15 g/kg body weight while the LD₅₀ for hamsters were 5-6 g/kg body weight. The latter was accompanied with degeneration of the proximal tubular cells, which correlated to increases in blood urea nitrogen and creatinine. Stevioside is excreted by the urine (Melis M. S. “Renal excretion of stevioside in rats”. J. Nat. Prod. 1992 May;55(5), p. 688-90) and is not metabolised in the isolated perfused rat liver (Ishii-Iwamoto E. L., Bracht A. “Stevioside is not metabolised in the isolated perfused rat liver”. Res. Commun. Mol. Pathol. Pharmacol. 1995 February;87(2), p. 167-75).

Stevioside and steviol showed no mutagenic effect on a number of Salmonella typhimurium strains (Klongpanichpak S., Temcharoen P., Toskulkao C., Apibal S., Glinsukon T. “Lack of mutagenicity of stevioside and steviol in Salmonella typhimurium TA 98 and TA 100”. J. Med. Assoc. Thai 1997 September;80 Suppl. 1, p. 121-128; Suttajit M., Vinitketkaumnuen U., Meevatee U., Buddhasukh D. “Mutagenicity and human chromosomal effect of stevioside, a sweetener from Stevia rebaudiana Bertoni”. Environ Health Perspect 1993 October;101 Suppl. 3, p. 53-56). In another study, it was confirmed that stevioside was not mutagenic whereas steviol, however, produced dose-related positive responses in some mutagenicity test (Matsui M., Matsui K., Kawasaki Y., Oda Y., Noguchi T., Kitagawa Y., Sawada M., Hayashi M., Nohmi T., Yoshihira K., Ishidate M. Jr., Sofuni T. “Evaluation of the genotoxicity of stevioside and steviol using six in vitro and one in vivo mutagenicity assays”. Mutagenesis 1996 November;11(6), p. 573-579).

Stevioside is not carcinogenic in F344 rats (Toyoda K., Matsui H., Shoda T., Uneyama C., Takada K., Takahashi M. “Assessment of the carcinogenicity of stevioside in F344 rats”. Food Chem. Toxicol. 1997 June;35(6), p. 597-603). Doses as high as 2.5 g/kg body weight/day had no effect on growth or reproduction in hamsters (Yodyingyuad V., Bunyawong S. “Effect of stevioside on growth and reproduction”. Hum. Reprod. 1991 January;6(1), p. 158-165).

To the knowledge of the inventors, no observations or reports showing potential toxic effects in humans have been published.

It will be obvious to the person skilled in the art that rearranged structures of the formula II are within the scope of the invention, and such rearrangements might occur naturally in the gastro intestinal tract. As example can be mentioned that rearrangement may occur at the C16 forming a double bond to the C15 and thereby leaving a single bond open for substitution at position 17. A COOH group at position 18 is open for a number of reactions such as reaction with alcohol, as well as a number of substituents can be provided at any point of the formula II structure. Also, other substituents such as e.g. saccharides, at the various C-atoms and the structures may be anticipated.

### EXAMPLES

In the following examples, the type II diabetic Goto-Kakizaki (GK) rats originated from Takeda Chemical Ind., Tokyo, Japan and were bred locally.

The normal Wistar rats and the NMRI mice were available from Bomholtg\dot{a}rd Breeding and Research Centre Ltd., Ry, Denmark.

The rats had a weight of 300-350 g and the mice a weight of 22-25 g. The animals were kept on a standard pellet diet and tap water ad libitum.

The stevioside is obtained from the Japanese company WAKO-TriCHEM.

The abbrevation IAUC means Incremental Area Under the Curve (above basal).

### Example 1

As examples of the effects of a compound including the chemical formulas II, stevioside was tested on normal Wistar rats and on GK rats. 2.0 g glucose/kg body weight and 0.2 g stevioside/kg body weight were dissolved in 0.9% saline and infused intravenously. The plasma glucose and insulin levels were measured over a period of 2 hours.

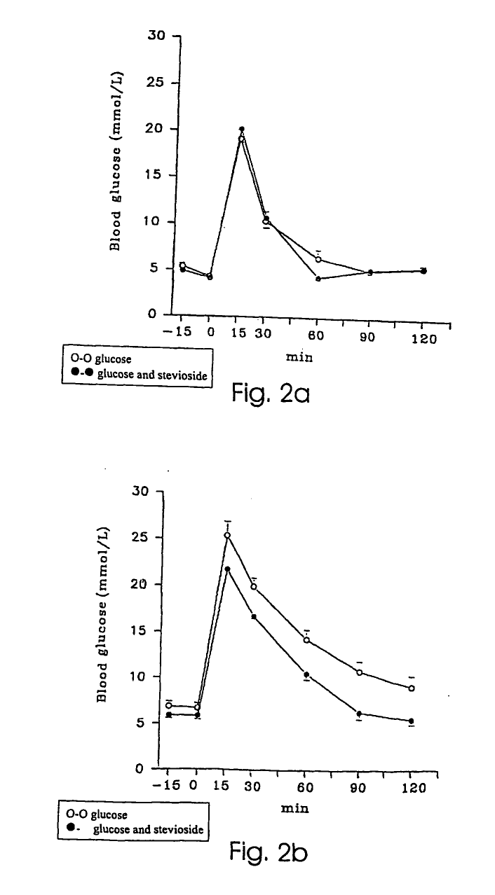

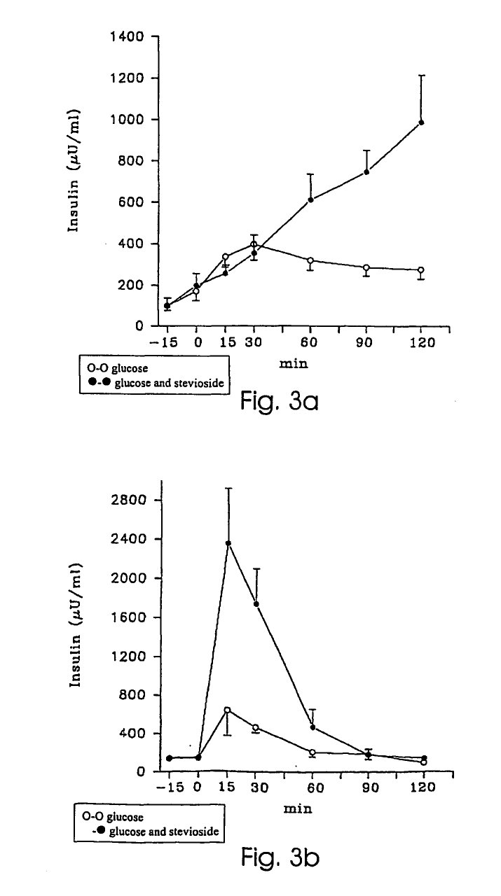

The results are shown in FIGS. 2a, 2b, 3a and 3b, were the O-O series (n=6 for Wistar and n=14 for GK) illustrate glucose infused alone and the \mathring{2}-\mathring{2} series (n=6 for Wistar and n=12 for GK) illustrate the combined glucose and stevioside infusion. Data are given as mean±SEM.

After administration of the glucose load, plasma glucose raised immediately and plasma insulin raised abruptly. When stevioside was added together with the glucose, a diminished glucose response was found in the GK-rat and a significant decrease was observed already after 30 min. In the GK rat, stevioside caused a pronounced increase in the insulin response compared to the Wistar rat. The stevioside-induced insulin response was delayed and increased throughout the whole test. The insulin response was monophasic.

This discovery of stevioside having a blood glucose reducing effect in the type II diabetic rat indicates that stevioside and compounds having a similar chemical structure can be used in a medicament for the treatment of NIDDM in man.

### Example 2

Islet from 6-10 NMRI mice were isolated and incubated in the presence of 16.7 mmol/l and 10⁻⁹-10⁻³ mol/l stevioside or 10⁻⁹-10⁻³ mol/l steviol.

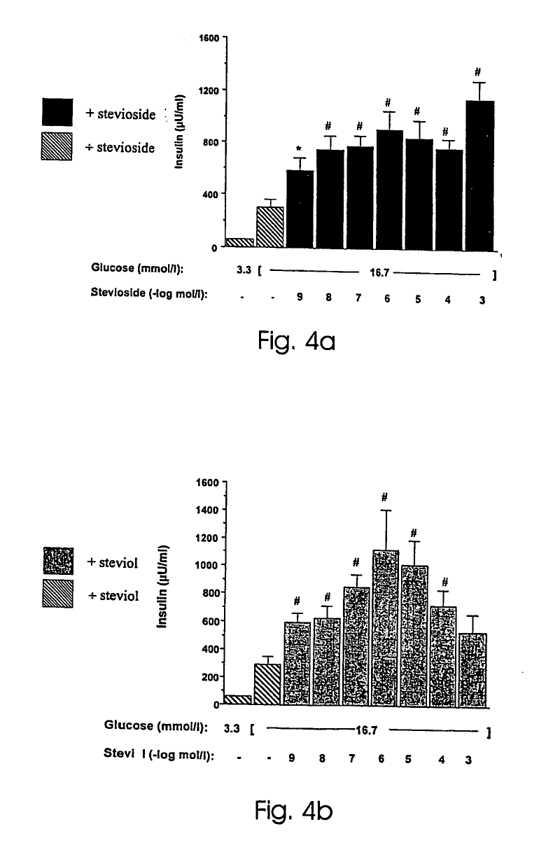

The results of these tests are illustrated in FIGS. 4a and 4b where each column represents mean±SEM from 24 incubations of single islets. Black bars in FIG. 4a indicate that stevioside is present and hatched bars indicate that stevioside is absent.

Grey bars in FIG. 4b indicate that steviol is present and hatched bars indicate that steviol is absent.

The figures show that stevioside and steviol are capable of potentiating glucose-stimulated insulin secretion. Further tests confirmed that a stimulatory effect was found already at a very low concentration (above 0.1 nM).

### Example 3

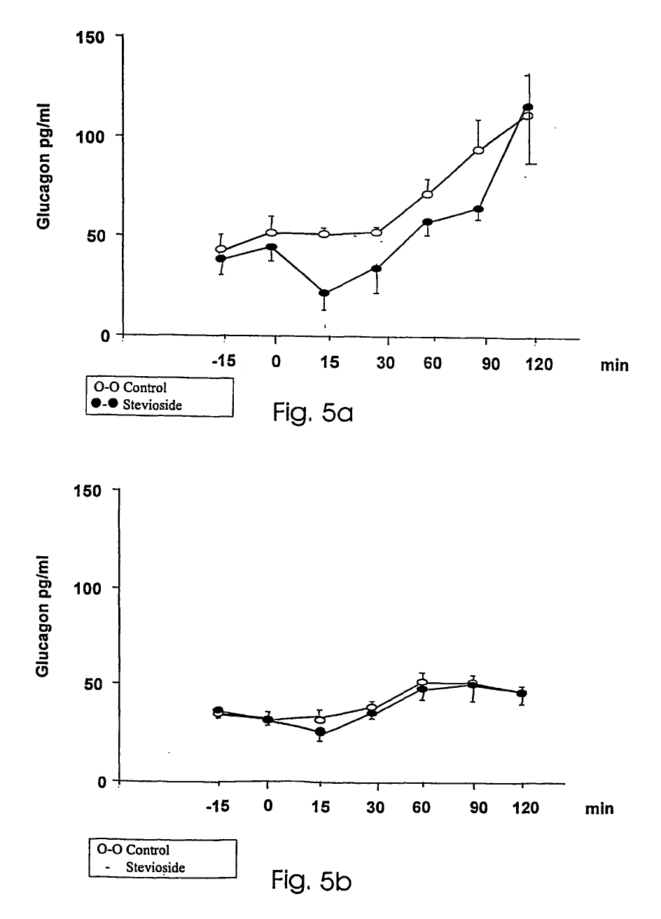

During a glucose tolerance test, an intravenous bolus of stevioside of 0.2 g/kg body weight was injected in GK rats (the \mathring{2}-\mathring{2} serie (n=6)). GK rats receiving 0.9% saline intravenously served as controls (the O-O serie (n=6)). Glucose 2.0 g/kg body weight was administered as a bolus at timepoint 0 min. The plasma glucagon responses are shown as mean±SEM in FIGS. 5a (control) and 5b (GK). The plasma glucagon was suppressed in the stevioside treated GK rat.

### Example 4

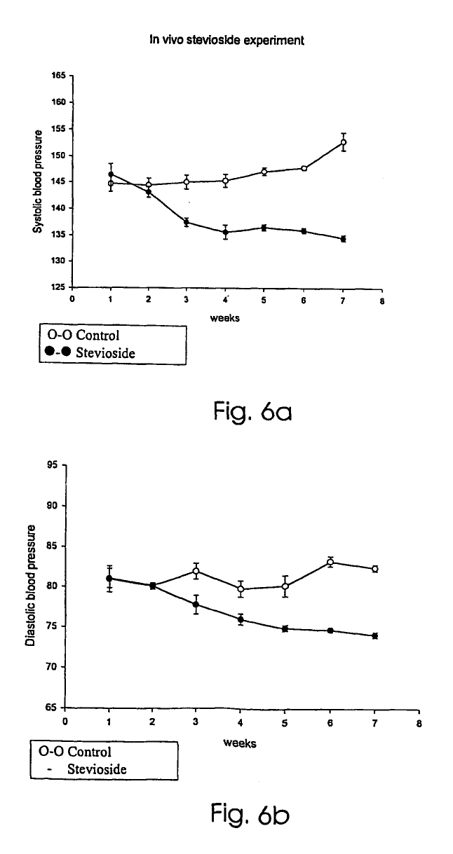

GK rats were treated with stevioside 0.025 g/kg body weight/24 h for 6 weeks. Stevioside was administered in the drinking water. GK rats receiving drinking water with 0.111 g D-glucose/kg body weight/24 h served as controls. Systolic (FIG. 6a, control: O-O series, stevioside-treated: \mathring{2}-\mathring{2} series) and diastolic (FIG. 6b, control: O-O series, stevioside-treated: \mathring{2}-\mathring{2} series) blood pressures were measured on the tail.

The figures show a 10-15% decrease in the blood pressure detectable after 2 weeks of treatment and the effect hereafter was stable and consistent during the study period.

### Example 5

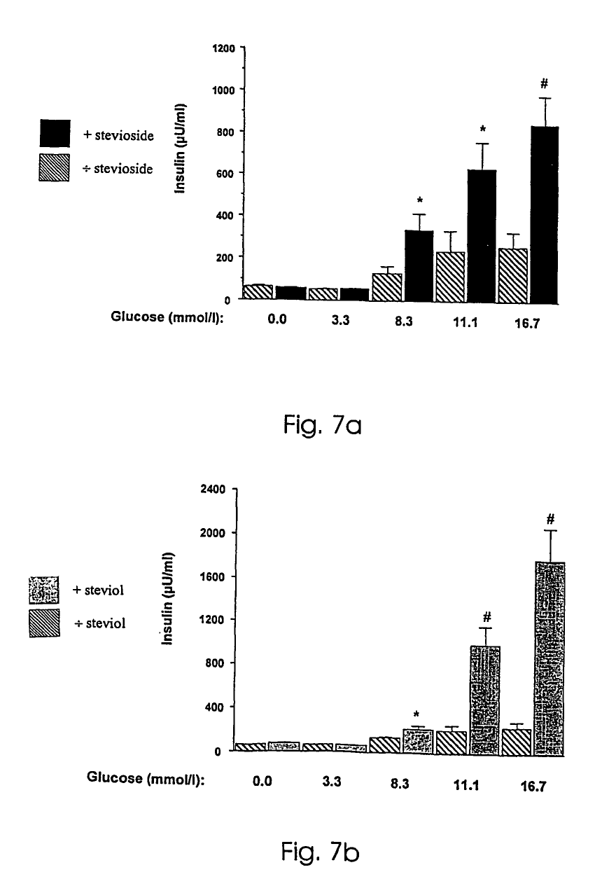

The influence of the maximal stimulatory doses of 10⁻³ mol/l stevioside and 10⁻⁶ mol/l steviol was studied in NMRI mouse islets over a range between 0 and 16.7 mmol/l glucose. Both stevioside (FIG. 7a) and steviol (FIG. 7b) potentiated insulin secretion at and above 8.3 mmol/l and indicated that the initiating level for stimulating insulin secretion was between 3.3 mmol/l and 8.3 mmol/l of glucose. Black bars in FIG. 7a indicate that stevioside is present and hatched bars indicate that stevioside is absent. Grey bars in FIG. 7b indicate that steviol is present and hatched bars indicate that steviol is absent.

### Example 6

Twenty type II diabetic patients (6 female/14 males) with a mean age of 63.6+7.5 years participated in a controlled randomised double blind crossover trial. They were supplemented for 6 weeks with soy protein for (50 g/day) with high levels of isoflavones (minimum 165 mg/day) and cotyledon fibers (20 g/day) or placebo (casein 5 μg/day) and cellulose (20 g/day) separated by a 3 week wash-out period.

This dietary supplement significantly reduced LDL-Cholesterol by 10% (p<0.05), LDL/HDL ratio by 12% (p<0.05), Apo B-100 by 30% (p<0.01), triglycerides by 22% (p<0.05) and homocystein by 14% (p<0.01). No change was observed in HDL-Cholesterol, Factor VIIc, von Willebrandt factor, fibrinogen, PAI-1, HbAlc or 24 hour blood pressure.

The results indicate beneficial effects of dietary supplementation with soy protein on cardiovascular risk markers in type II diabetic subjects. The improvement is also seen in individuals with near-normal lipid values. Ingestion of soy product has been shown to further improve the effectiveness of low-fat diets in non-diabetic subjects and the dietary supplementation in type II diabetic patients may provide an acceptable and effective option for blood lipid control, thereby postponing or even preventing drug therapy.

### Example 7

Twelve type II diabetic patients (4 female/8 males) with a mean age of 65.8±1.6 years, a diabetes duration of 6.0±1.3 years, a mean body mass index of 28.5±1.0, and a mean glycated hemoglobin HbAlc of 7.4+0.4 percent were included in the study.

The experiment was an acute, paired, cross-over study in which two test meals were served during the experiments (A: Standard meal supplemented with 1 g of stevioside given orally; B: Standard meal given together with 1 g of gelatine (placebo) given orally. The total energy content of the test meals was 1725 kJ (protein 16 E %, fat 30 E %, carbohydrate 54 E %).

Blood samples were drawn from an antecubital vein 30 minutes before and 240 minutes after ingestion of the test meal. The arterial blood pressure was continuously monitored during the experiment. Students paired t-test was used for comparing the effects of stevioside with placebo on the parameters measured. Data are given as mean±SEM.

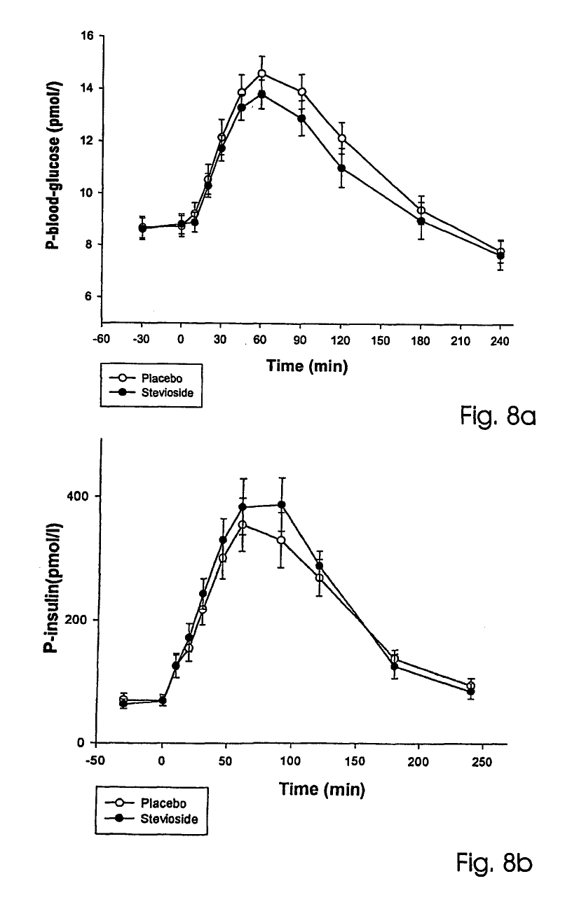

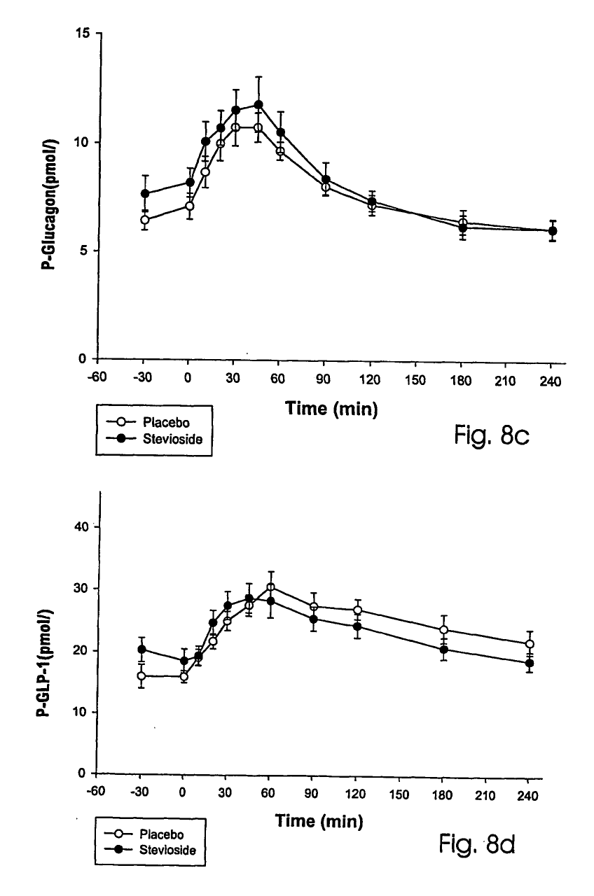

Stevioside reduced the postprandial blood glucose response by 18±5% (p<0.004) compared to placebo (absolute IAUC 638±55 vs. 522±64 mmol/l x 240 min; p<0.02) as seen in FIG. 8a. Stevioside tended to stimulate the insulin response in type II diabetic patients (enhance the area under the insulin response curve (IAUC)), however the difference did not reach statistical significance (p=0.09) (FIG. 8b).

Stevioside significantly reduced the postprandial glucagon levels compared to placebo (348±46 vs. 281±33; p=0.02) (FIG. 8c).

Stevioside significantly reduced the postprandial glucagon like peptide-1 (GLP-1) levels compared to placebo (2208±253 vs. 1529±296; p<0.045) (FIG. 8d).

### Example 8

In the following example is the term “Soy” defined as a composition comprising 65 weight-% soy protein, at least 0.2 weight-% isoflavones and 18 weight-% soy dietary fibres, mostly cotyledon fibres. “Altromin” is a standard carbohydrate rich laboratory animal diet.”

Four test diets, A, B, C and D as defined below, were administered for four weeks to four groups of adult rats.

A: Altromin; n=12 (Alt).

B: Altromin supplemented with stevioside; n=12; (Alt+Ste).

C: 80% Soy plus 20% Altromin; n=12; (Soy).

D: 80% Soy plus 20% Altromin plus stevioside; n=12; (Soy+Ste)

The four above mentioned diets all have the same amount of vitamins and minerals.

Each experimental group consisted of twelve female Goto-Kakizaki with an age of 9 weeks. The rats received the stevioside (0.025 g/kg body weight/day) with the drinking water.

By the end of the third experimental week intra-arterial catheters were implanted into the carotid artery thereby enabling blood sampling during a 240 minutes glucose-tolerance test which was carried out by the end of the experiment at week 4. Blood samples were drawn after a bolus infusion of 2.0 g D-glucose/kg body weight.

Plasma concentrations of glucose, insulin, and glucagon were measured during the glucose tolerance test. Immediately before the glucose tolerance test fasting levels of triglycerides and cholesterol were determined. Concomitantly, the systolic blood pressure was measured using a tail cuff.

Effects on Plasma-Glucose:

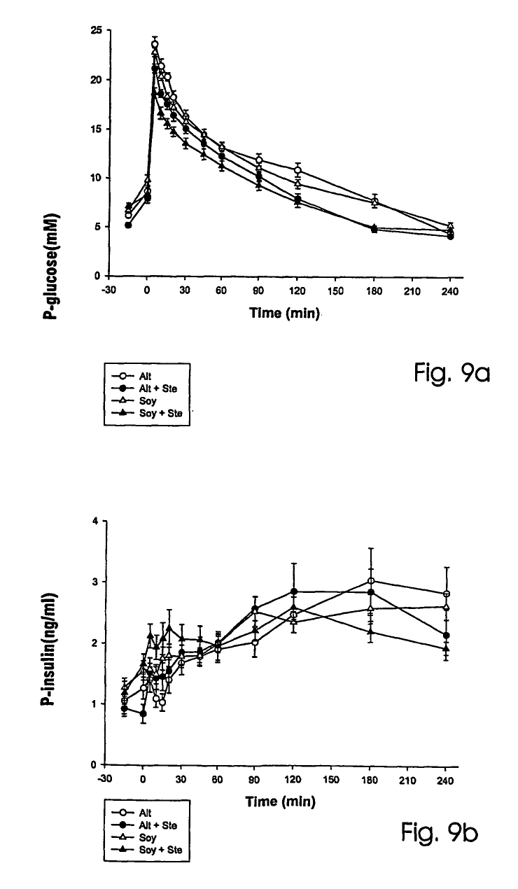

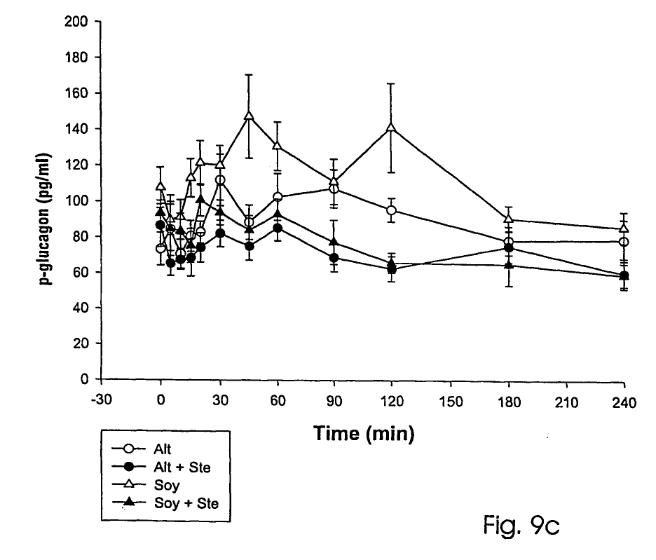

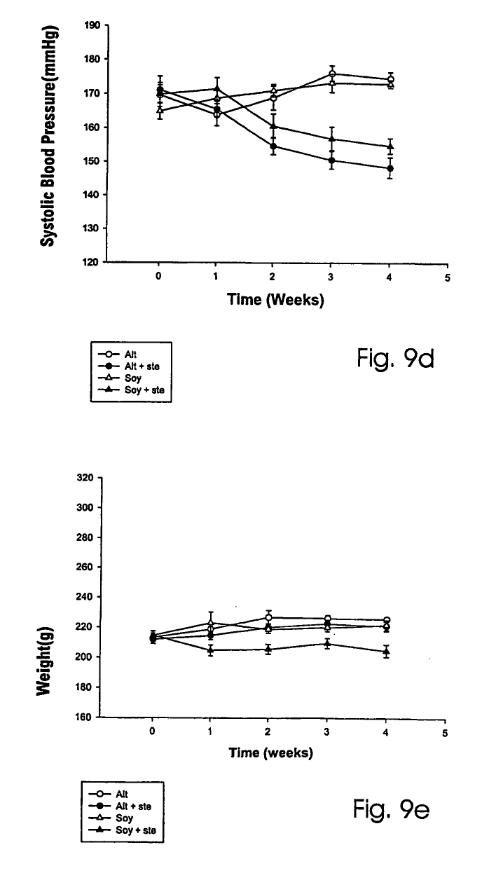

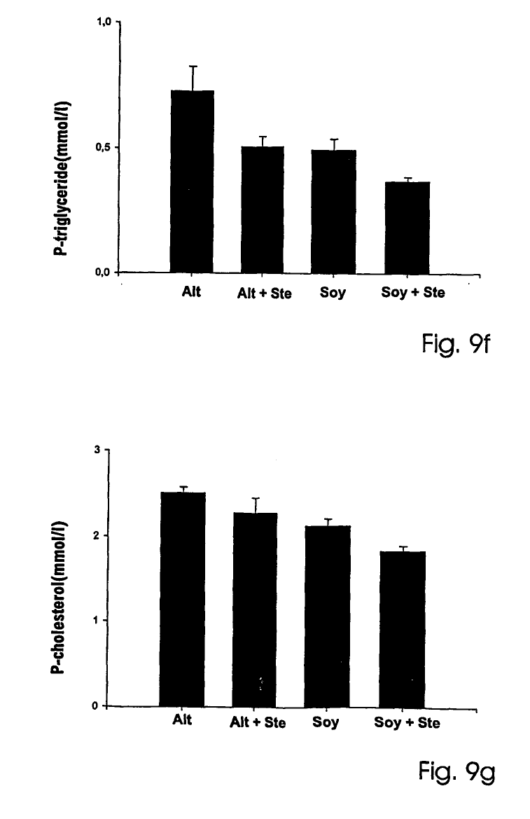

As seen at FIG. 8 and in Table I below stevioside reduced the incremental area (IAUC) under the glucose response curve during the glucose tolerance testing both in the Altromin (p<0.05) and in the soy+20% Altromin group (Soy) (p<0.001). The relative effect of stevioside was more pronounced in the group receiving soy+20% Altromin group compared to the group receiving Altromin. The combination of soy and stevioside synergistically reduced the area under the glucose response curve compared to the Altromin group (p<0.0001) (FIG. 9a.).

(Plasma glucose was measured using MPR 3, 166 391, Glucose/GOD-PAP Method from Boehringer Mannheim)

Effects on Plasma Insulin:

The group receiving soy+stevioside (Soy+Ste) has reduced incremental area under the insulin response curve compared to the Altromin+stevioside group (Alt+Ste) as seen at FIG. 9 and in Table I below. Considering the concomitant blood glucose responses this indicates that soy increases the insulin sensitivity. Stevioside did not alter the insulin responses in the Altromin and soy diets when studying the total response curve from 0 to 240 minutes. However, in both groups supplementation of the diets with stevioside significantly improved the first phase insulin responses which is subdued as a characteristic feature of type II diabetes. The combination of soy+stevioside synergistically improved the first phase insulin response (p<0.05) (FIG. 9b).

(Plasma insulin was measured using Sensitive Rat Insulin RIA, Cat # SRI-13K from Linco)

Effects on Plasma Glucagon:

Stevioside significantly reduced the area under the plasma-glucagon response curve during the glucose tolerance test in both the groups receiving Altromin (p<0.003) and soy (p<0.01) (see FIG. 9c and Table I below).

(Plasma glucagon was measured using Glucagon RIA, Cat # GL-32K from Linco)

Effects on Blood Pressure:

A marked significant suppression of the systolic blood pressure (p<0.05) (Table I) is elicited by stevioside in combination with either Altromin (Δ=−28 mmHg) or soy (Δ=−21 mmHg) as depicted in FIG. 9d.

(Blood pressure was measured using TSE Non-Invasive Blood Pressure Monitoring System from Technical Scientific Equipment GmbH)

Effects on Body Weight:

The initial weights in the four groups did not differ (FIG. 5). Apparently the combination of soy and stevioside prevented weight gain as seen in FIG. 9e.

Effects on Triglyceride and Cholesterol.

Stevioside causes a significant suppression of the fasting triglyceride levels in combination with either Altromin (p<0.05) or soy (p<0.02) (Table I). Soy significantly reduced the fasting triglyceride levels with or without supplementation of stevioside (p<0.05 and p<0.002, respectively) (Table I). Stevioside given in combination with soy synergistically reduced the fasting total cholesterol levels compared to diets containing Altromin alone (p<0.0001). Soy alone also reduced the total cholesterol levels compared to Altromin alone (p<0.002) (FIG. 9f. and FIG. 9g) (Table I).

(Plasma cholesterol was measured GOD-PAP from Roche and triglycerides was measured using GHOD-PAP from Roche)

Stevioside exerts beneficial effects in type II diabetes i.e. reduces blood glucose, suppresses glucagon and improve first phase insulin secretion. The results also indicates that soy improves insulin sensitivity, a characteristic feature of the metabolic syndrome. Stevioside exerts a pronounced blood pressure reduction both with as well as without the presence of soy. The combination of stevioside and soy has a synergistic suppressive effect on blood glucose levels, enhances first phase insulin secretion, supresses fasting plasma triglyceride and total cholesterol and the combination of soy and stevioside seems to prevent weight gain. The combination of stevioside and soy appears to possess the potential of an effective treatment of a number of the characteristic features of the metabolic syndrome i.e. type II diabetes, hypertension, dyslipidemia and obesity.

TABLE I
Areas under the p-glucose, -insulin and -glucagon response curves during
the glucose tolerance test in the four experimental groups. Change in systolic blood pressure at
start and at end of the study period. Fasting plasma- triglyceride and -total cholesterol
concentrations by the end of the study.

| | | IAUC | IAUC | | | | |
|---|---|---|---|---|---|---|---|
| | IAUC | p-insulin | p-insulin | IAUC | Change in blood | | |
| | p-glucose | (ng/ml × | (ng/ml × 30 | p-glucagon | pressure (mmHg) | Triglycerides | Cholesterol |
| Group | (mM × 240 min) | 240 min) | min) | (pg/ml × 240 min) | From week 0 to 4 | (mM) | (mM) |
| Altromin | 991 ± 96 | 317 ± 55 | 11 ± 4 | 21918 ± 1467 | 5 ± 4 | 0.72 ± 0.10 | 2.51 ± 007 |
| Altromin + | 757 ± 53 | 375 ± 42 | 19 ± 4 | 17023 ± 1449 | −23 ± 6 | 0.50 ± 0.04 | 2.28 ± 0.18 |
| Stevioside | | | | | | | |
| Soy + 20% Altr min | 820 ± 75 | 218 ± 22 | 9 ± 2 | 26200 ± 2410 | 8 ± 3 | 0.49 ± 0.04 | 2.13 ± 0.08 |
| Soy + 20% Altromin + | 439 ± 56 | 248 ± 27 | 24 ± 5 | 17229 ± 1819 | −13 ± 5 | 0.37 ± 0.02 | 1.84 ± 0.06 |
| Stevi side | | | | | | | |

What is claimed is:

1. A composition comprising,
(a) a soy protein source,
(b) at least one phytoestrogen compound,
(c) dietary fibres, and
(d) a substance selected from the group consisting of steviol, isosteviol, glucosteviol, gymnemic acid, steviolbioside, stevioside, Rebaudioside A, Rebaudioside B, Rebaudioside C, Rebaudioside D, Rebaudioside E or Dulcoside A, their pharmaceutically acceptable analogues or their pharmaceutically acceptable derivates

2. A composition according to claim 1, wherein the soy protein source is selected from isolated soy protein, soy protein concentrate, or soy flour, said soy protein source providing an amount of soy protein, which is at least 45 weight percent of the total protein content of the composition, said total protein content providing at least 15 percent of the total energy content of the composition.

3. A composition according to claim 1, wherein the at least one phytoestrogen compound are present in an amount of more than 0.10 weight percent of the soy protein content of the composition.

4. A composition according to claim 1, wherein the dietary fibres are present in an amount of more than 4 weight percent of the total weight of the nutritional composition on a dry basis.

5. A composition according to claim 4, wherein the dietary fibres are soybean fibres.

6. A composition according to claim 5, wherein the soybean fibres are soy cotyledon fibres.

7. A composition according to claim 1, wherein the phytoestrogen compound is selected among isoflavones.

8. A composition according to claim 7 wherein the isoflavones are selected from the group consisting of genistein, daidzein, glycitein and equol.

9. A composition according to claim 7, wherein some or all of the isoflavones are present in the aglycone form.

10. A composition according to claim 1 in the form of a micronutrient.

11. A composition according to claim 1 in the form or a functional food ingredient.

12. A composition according to claim 1 in the form of a special dietary supplement.

13. A composition according to claim 1 in the form of a medicament.

14. A method for treating type 2 diabetes in a subject which comprises administering a therapeutically effective amount of the composition of claim 1 to a subject in need of such treatment.

15. A method according to claim 14 wherein the composition is administered to provide improved glucose tolerance, an increased insulin sensitivity, reduced serum glucose levels or an improved insulin secretion.

16. A method for reducing the influx of cholesterol into the arterial wall in a diabetic subject which comprises administering a therapeutically effective amount of the composition of claim 1 to a subject in need of such treatment.

17. A method for reducing the influx of triglycerides into the arterial wall in a diabetic subject which comprises administering a therapeutically effective amount of the composition of claim 1 to a subject in need of such treatment.

18. A method for treating a cardiovascular disease in a diabetic subject which comprises administering a therapeutically effective amount of the composition of claim 1 to a subject in need of such treatment.

19. A method according to claim 18 wherein said cardiovascular disease is selected from the group consisting of hypertriglyceridaemia, hypercholesterolaemia, hypertension, hyperglycaemia, hyperinsulinaemia, arteriosclerosis, atherosclerosis, arteriolosclerosis, angina pectoris, thrombosis and myocardial infarction.

20. A pharmaceutical preparation comprising a composition according to claim 1 and optionally including a pharmaceutically acceptable carrier for the composition.

21. A method for manufacturing a medicament for treating a subject suffering of a disease selected from the group consisting of type 2 diabetes, the metabolic syndrome, a cardiovascular disease, obesity, hypertension and dyslipidemia, which comprises formulating as the medicament, a copmposition according to claim 1.

22. The method according to claim 21 wherein the medicament is effective in treating effects in a diabetic subject selected from the group consisting of lowering serum glucose levels, lowering total serum cholesterol levels, lowering serum LDL-cholesterol levels, lowering serum triglyceride levels, lowering serum homocystein levels, lowering blood pressure, increasing the HDL/LDL-cholesterol ratio and increasing serum HDL-cholesterol levels.
